(19) United States
(12) Patent Application Publication
(10) Pub. No.: US 2022/0133616 A1
(43) Pub. Date: May 5, 2022

(54) Compositions, Methods and Uses for Treating Skin Aging

(71) Applicant: Robin James Evans, Thousand Oaks, CA (US)

(72) Inventor: Robin James Evans, Thousand Oaks, CA (US)

(21) Appl. No.: 17/520,535

(22) Filed: Nov. 5, 2021

Related U.S. Application Data
(60) Provisional application No. 63/110,286, filed on Nov. 5, 2020.

(51) Int. Cl. A61K 8/73; A61Q 19/08; A61K 8/31; A61K 8/34

(52) U.S. Cl. CPC A61K 8/735; A61Q 19/08; A61K 2800/592; A61K 8/34; A61K 8/31

(57) ABSTRACT

The present specification discloses compositions comprising one or more monoterpene compounds and HA polymers as well as methods and uses of such compositions for treating a soft tissue condition of an individual and/or cellular senescence in a skin region of an individual.

This application claims the benefit of priority and is entitled to the filing date pursuant to 35 U.S.C. § 119(e) of U.S. Provisional Patent Application 63/110,286, filed Nov. 5, 2020, the content of which is hereby incorporated by reference in its entirety.

The skin is the outer covering of a mammalian body. Typically, the largest organ of the integumentary system, skin is composed of three primary layers: the epidermis, the dermis and the hypodermis. The epidermis is the outermost epithelial layer of the skin and comprises stratified, squamous epithelium that is generated from keratinocytes located in the basement or basal layer. This layer contains no blood or lymphatic vessels. The epidermis forms the waterproof, protective wrap over the body's surface which also serves as a barrier to infection. The dermis is an epithelial layer of skin beneath the epidermis, comprises an extracellular matrix (ECM) with collagen, elastin, and glycosaminoglycans such as hyaluronic acid, and serves to cushions the body from stress and strain. Besides fibroblasts, which create the ECM, the dermis contains blood vessels, nerves and lymphatic channels. Skin appendages (hair, sebaceous glands, sweat glands and nails) are also anchored within the dermis. The hypodermis lies below the dermis and comprises loose connective tissue, adipose tissue and elastin. This layer is critical in cellular signaling as well as contributing to the youthful appearance of an individual. Although not composed of epithelial tissue, and thus not part of the skin, the hypodermis attaches the skin to underlying bone and muscle as well as supplying it with blood vessels and nerves.

Our skin creates an active barrier against our external environment and guards the underlying muscles, bones, ligaments and internal organs. This organ governs thermoregulation of the body, prevention of water loss, and protection against pathogens as both a physical barrier as well as through immune regulation. As the external part of an individual's body, the skin also serves an important cosmetic/social function as the appearance, beauty and/or health of the skin affects an individual's self-esteem as well as his/her interaction with others.

Mammalian skin changes drastically with age resulting in a loss of maximal function and reserve capacity. A complex process affecting all layers, aging causes loss of volume, elasticity, firmness and resiliency of the skin due to the thinning of the epithelial layers of the epidermis and dermis, and degeneration of the underlying fat and connective tissue of the hypodermis. These atrophic changes affect all skin areas, including facial skin, neck skin, chest skin, breast skin, hand skin, leg skin, and feet skin. Symptoms associated with skin aging include, e.g., wrinkles, fine lines, thinning skin, sagging skin, sinking skin, skin dryness, skin roughness, and skin itchiness.

In an effort to treat or correct the effects of aging, soft tissue fillers have been developed and widely used to address the symptoms of skin aging. Such fillers replace lost endogenous matrix polymers, or enhance/facilitate the function of existing matrix polymers, and have been used in cosmetic applications to fill wrinkles, plump thin lips, fill-in sunken eyes or shallow cheeks and otherwise restore a smoother, more youthful appearance. Unfortunately, the beneficial efforts of current soft tissue fillers are temporarily because they merely address the symptoms and not the causes of skin aging.

The present specification discloses compositions, methods and uses to treat a skin condition caused by the effects of aging by reducing cellular senescence in skin cells by enhancing autophagy through the modulation of the mammalian (now “mechanistic”) target of rapamyacin (mTOR) signaling pathway.

### SUMMARY

Aspects of the present specification disclose compositions comprising one or more monoterpene compounds and HA polymers. The disclosed one or more monoterpene compounds include a monoterpene, a derivative of a monoterpene, a monoterpenoid, a derivative of a monoterpenoid, or any combination thereof. The disclosed HA polymers can be low molecular weigh HA polymers, high molecular weight HA polymers, or both and can comprise mixtures of uncrosslinked and crosslinked HA polymers.

Other aspects of the present specification disclose methods of treating a soft tissue condition of an individual. The disclosed methods comprise administering a composition disclosed herein to a skin region of the individual.

Other aspects of the present specification disclose compositions disclosed herein for use in treating a soft tissue condition of an individual, use of compositions disclosed herein for treating a soft tissue condition of an individual, and use of compositions disclosed herein in the manufacture of a medicament for the treatment a soft tissue condition of an individual.

Other aspects of the present specification disclose methods of treating cellular senescence in a skin region of an individual. The disclosed methods comprise administering a composition disclosed herein to a skin region of the individual.

Other aspects of the present specification disclose compositions disclosed herein for use in treating cellular senescence in a skin region of an individual, use of compositions defined herein for treating cellular senescence in a skin region of an individual, and use of compositions disclosed herein in the manufacture of a medicament for the treatment cellular senescence in a skin region of an individual.

### DETAILED DESCRIPTION

A progressive phenomenon, skin aging can be considered the result of two independent processes: extrinsic and intrinsic aging. Extrinsic aging is a result of accumulated environmental damage to skin cells with the primary causes being sun exposure, air pollution, alcohol consumption, tobacco use, and poor nutrition. Such aging occurs through the generation of free radicals and reactive oxygen species (ROS) secondary to cellular damage. ROS activate the MAPK pathway and subsequently increase inflammation through MMP production. This results in degradation of collagen and subsequent formation of wrinkles, pigment dysregulation, and dry skin. Elastin also begins to accumulate and degrade in the deep dermis. The normal collagen to elastin ratio is lost. The pathologic change in elastin is termed “solar elastosis” and is significant evidence of extrinsic skin aging. The number of melanocytes decreases 8-20% per decade after the age of 30¹. Accumulation of ROS causes pathologic changes in remaining melanocytes. Foci of activated melanocytes form lentigines (“age spots”) and foci of inactivated melanocytes cause amelanosis and gray hair.

Intrinsic aging is related to chronological changes that are due in part to genetic or epigenetic factors that cause cellular senescence. Cellular senescence is permanent cell cycle arrest in damaged cells resulting in arrest of growth, resistance to apoptosis and altered differentiation functions. It has been shown to be produced by cellular stress, mitochondrial dysfunction, chemotherapy, radiation therapy, and oncogene activation. Induction of senescence is thought to prevent the damage to the next generation of cells and reduces the risk of malignant transformation. However, with senescence comes a loss of regenerative capacity, otherwise known as aging. Referred to as antagonistic pleiotropy, this functional dichotomy underscores the importance of cellular senescence to protect against cancer early in life but detrimental as the organism ages.

Although the cell cycle is arrested in cellular senescence, the cell continues to be metabolically active and secrete pro-inflammatory factors (IL-6, IL-8), chemokines (CXCR2), matrix metalloproteinases (MMP3 and MMP9) and growth factors (IGFBP7). Named senescence-associated secretory phenotype (SASP), senescent cells create a pro-inflammatory microenvironment that detrimentally impacts surrounding cells causing intrinsic skin aging due to cell damage as well as increased susceptibility to tumorigenesis and chemotherapy resistance. Increased accumulation senescent cells due to aging results in increased SASP, chronic inflammation and altered immune function. Termed inflammaging this approach suggests that to reduce or reverse aging is synonymous with reducing the pathways of chronic inflammation itself.

Autophagy is the process of cellular self-digestion. This is performed by delivering cytoplastic material to the lysosome for breakdown. There are three known types of autophagy: macroautophagy, microautophagy and chaperone-mediated autophagy. Macroautophagy uses autophagy-related proteins (ATGs) and is characterized by autophagosome formation. An autophagosome is a double-membraned vesicle within the cytoplasm that fuses with lysosomes to degrade its contents. Microautophagy, in mammals, is an invagination of the late endosomal membrane to trap cytoplastic material which is then degraded. Finally, chaperone-mediated autophagy specifically relies on the cytosolic chaperone hsc70 for specific substrate targeting to the lysosome.

Autophagy, and its regulation, plays a critical role in skin homeostasis by identifying and removing senescent cells both in the epithelium and dermis. As such, one mechanism by which cellular senescence can be regulated is through autophagy. The present specification discloses that a monoterpene compound can reverse cellular senescence by regulating autophagy. Such reversal is mediated by the ability of monoterpene compound to de-phosphorylate mTOR, eukaryotic initiation factor 4E (elF4E), and elF4E binding protein-1 (4E-BP1).

mTOR is a master controller of cellular function. It is a serine/threonine protein kinase in the PI3K-related kinase (PIKK). mTOR integrates environmental and intracellular nutrient growth factor signals to effect cell growth, proliferation, cell survival and inflammation. Importantly, mTOR is comprised of two distinct protein complexes: mTOR Complex 1 (mTORC1) and 2 (mTORC2). mTORC1 consists of mTOR, Raptor, GβL (mammalian lethal with SEC13 protein 8) and domain-containing mTOR-interacting protein (DEPTOR). mTORC1 modulates cap-dependent translation in response to nutrients, hormones and growth factors. On the other hand, mTORC2 regulates cytoskeletal dynamics, ion transport and growth.

The present specification discloses compositions comprising a monoterpene compound and hyaluronic acid polymers. In some embodiments, a composition disclosed herein is an admixture and/or coformulation of a monoterpene compound and hyaluronic acid polymers. In some embodiments, a composition disclosed herein is conjugated comprising a monoterpene compound and hyaluronic acid polymers, referred to as a monoterpene compound-HA polymer conjugate.

A monoterpene compound includes a monoterpene, a derivative of a monoterpene, a monoterpenoid, and a derivative of a monoterpenoid. Monoterpenes are a class of terpenes that consist of two isoprene units and have the molecular formula C₁₀H₁₆. Monoterpenes are derived biosynthetically from activated forms of isoprene, such as, e.g., isopentenyl pyrophosphate (IPP or also isopentenyl diphosphate) and dimethylallyl pyrophosphate (DMAPP or also dimethylallyl diphosphate). A disclosed monoterpene may be linear (acyclic) or contain rings (monocyclic, bicyclic, and trycyclic) Non-limiting examples of a linear monoterpene include α-myrcene, β-myrcene, α-ocimene, and β-ocimene. Non-limiting examples of a monocyclic monoterpene include limonene, α-phellandrene, β-phellandrene, α-terpineol, β-terpineol, γ-terpineol, 4-terpineol, terpinen-4-ol, α-terpinene, β-terpinene, γ-terpinene, and δ-terpinene (terpinolene). Non-limiting examples of a bicyclic monoterpene include camphene, carene, myrtersol, myrtenal, α-pinene, β-pinene, pinocarveol, sabinene, thujene, verbanol, and verbanon. Non-limiting examples of a tricyclic monoterpene include tricyclene.

Monoterpenoids are monoterpenes that have been modified chemically, such as by oxidation, demethylation, or rearrangement of the carbon skeleton. Non-limiting examples of a linear monoterpenoid include citral, citronellal, citronellol, geranial (citral A), geraniol, geranyl pyrophosphate, halomon, linalool, myrcenol, and nerol (citral B). Non-limiting examples of a monocyclic monoterpenoid include carvacrol, p-cymene, grapefruit mercaptan, menthol, perillyl alcohol (s(−)) and (r(+)), α-thujaplicin, β-thujaplicin (hinokitiol), γ-thujaplicin, and thymol. Non-limiting examples of a bicyclic monoterpenoid include ascaridole, borneol, bornyl acetate, camphor, eucalyptol, and umbellulone.

A derivative of a monoterpene or monoterpenoid disclosed herein is a monoterpene or monoterpenoid modified to include a functional group. Non-limiting examples of a derivative of monoterpene or monoterpenoid include carbamates, esters, ethers, alcohols and aldehydes of a monoterpene or monoterpenoid disclosed herein. A monoterpene or monoterpenoid alcohol may be derivatized to a carbamate, an ester, an ether, an aldehyde or an acid. Esters of a monoterpene or monoterpenoid can be derived from an inorganic acid or an organic acid. Inorganic acids include, but are not limited to, phosphoric acid, sulfuric acid, and nitric acid. Organic acids include, but are not limited to, carboxylic acid such as benzoic acid, fatty acid, acetic acid and propionic acid, and any other agent bearing at least one carboxylic acid functional group. Examples of esters of monoterpene or monoterpenoid alcohols include, but are not limited to, carboxylic acid esters (such as benzoate esters, fatty acid esters (e.g., palmitate ester, linoleate ester, stearate ester, butyryl ester and oleate ester), acetates, propionates (or propanoates), and formates), phosphates, sulfates, and carbamates (e.g., N,N-dimethylaminocarbonyl).

In some embodiments, a monoterpenoid derivative is a perillyl alcohol derivative. The derivatives of perillyl alcohol include, perillyl alcohol carbamates, perillyl alcohol esters, perillic aldehydes, dihydroperillic acid, perillic acid, perillic aldehyde derivatives, dihydroperillic acid esters and perillic acid esters. The derivatives of perillyl alcohol may also include its oxidative and nucleophilic/electrophilic addition derivatives. Perillyl alcohol and its derivatives are also described in U.S. Pat. Nos. 3,957,856, 4,306,099, 7,884,252, 8,236,863, 8,507,734, 8,916,545, 9,499,461, 9,580,372, 9,663,428, and 10,092,562, the content of each of which is hereby incorporated by reference.

In aspects of these embodiments, perillyl alcohol (POH) derivatives may be perillyl alcohol fatty acid esters, such as palmitoyl ester of POH and linoleoyl ester of POH, the chemical structures of which are shown below.

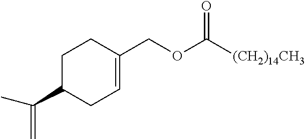

Hexadecanoic acid 4-isopropenyl-cyclohex-1-enylmethyl ester (palmitoyl ester of POH)

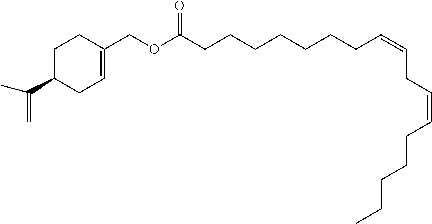 [SMILES: C=C(C)[C@@H]1CC=C(COC(=O)CCCCCCC/C=C\C/C=C\CCCCC)CC1]

Octadeca-9,12-dienoic acid 4-isopropenyl-cyclohex-1-enylmethyl ester (linoleoyl ester of POH)

In some embodiments, a composition disclosed herein comprises a monoterpene compound in an amount necessary and sufficient to improve a skin condition as disclosed herein. In aspect of these embodiments, a composition disclosed herein comprises a monoterpene compound in an amount of, e.g., about 0.1%, about 0.2%, about 0.3%, about 0.4%, about 0.5%, about 0.6%, about 0.7%, about 0.8%, about 0.9%, or about 1.0% by weight of the composition. In other aspects of these embodiments, a composition disclosed herein comprises a monoterpene compound in an amount of, e.g., at least 0.1%, at least 0.2%, at least 0.3%, at least 0.4%, at least 0.5%, at least 0.6%, at least 0.7%, at least 0.8%, at least 0.9%, or at least 1.0% by weight of the composition. In yet other aspects of these embodiments, a composition disclosed herein comprises a monoterpene compound in an amount of, e.g., at most 0.1%, at most 0.2%, at most 0.3%, at most 0.4%, at most 0.5%, at most 0.6%, at most 0.7%, at most 0.8%, at most 0.9%, or at most 1.0% by weight of the composition. In still other aspects of these embodiments, a composition disclosed herein comprises a monoterpene compound in an amount of, e.g., about 0.1% to about 0.3%, about 0.1% to about 0.5%, about 0.1% to about 0.7%, about 0.1% to about 1.0%, about 0.3% to about 0.5%, about 0.3% to about 0.7%, about 0.3% to about 1.0%, about 0.5% to about 0.7%, about 0.5% to about 1.0%, about 0.7% to about 1.0% by weight of the composition.

In aspect of these embodiments, a composition disclosed herein comprises a monoterpene compound in an amount of, e.g., about 5 mM/L, about 10 mM/L, about 15 mM/L, about 20 mM/L, about 25 mM/L, about 30 mM/L, about 35 mM/L, about 40 mM/L, about 45 mM/L, about 50 mM/L, about 55 mM/L, about 60 mM/L, about 65 mM/L, about 70 mM/L, about 75 mM/L, about 80 mM/L, about 85 mM/L, about 90 mM/L, about 95 mM/L, or about 100 mM/L. In other aspect of these embodiments, a composition disclosed herein comprises a monoterpene compound in an amount of, e.g., at least 5 mM/L, at least 10 mM/L, at least 15 mM/L, at least 20 mM/L, at least 25 mM/L, at least 30 mM/L, at least 35 mM/L, at least 40 mM/L, at least 45 mM/L, at least 50 mM/L, at least 55 mM/L, at least 60 mM/L, at least 65 mM/L, at least 70 mM/L, at least 75 mM/L, at least 80 mM/L, at least 85 mM/L, at least 90 mM/L, at least 95 mM/L, or at least 100 mM/L. In yet other aspect of these embodiments, a composition disclosed herein comprises a monoterpene compound in an amount of, e.g., at most 5 mM/L, at most 10 mM/L, at most 15 mM/L, at most 20 mM/L, at most 25 mM/L, at most 30 mM/L, at most 35 mM/L, at most 40 mM/L, at most 45 mM/L, at most 50 mM/L, at most 55 mM/L, at most 60 mM/L, at most 65 mM/L, at most 70 mM/L, at most 75 mM/L, at most 80 mM/L, at most 85 mM/L, at most 90 mM/L, at most 95 mM/L, or at most 100 mM/L.

In still other aspect of these embodiments, a composition disclosed herein comprises a monoterpene compound in an amount of, e.g., about 5 mM/L to about 10 mM/L, about 5 mM/L to about 20 mM/L, about 5 mM/L to about 30 mM/L, about 5 mM/L to about 40 mM/L, about 5 mM/L to about 50 mM/L, about 5 mM/L to about 60 mM/L, about 5 mM/L to about 70 mM/L, about 5 mM/L to about 80 mM/L, about 5 mM/L to about 90 mM/L, about 5 mM/L to about 100 mM/L, about 10 mM/L to about 20 mM/L, about 10 mM/L to about 30 mM/L, about 10 mM/L to about 40 mM/L, about 10 mM/L to about 50 mM/L, about 10 mM/L to about 60 mM/L, about 10 mM/L to about 70 mM/L, about 10 mM/L to about 80 mM/L, about 10 mM/L to about 90 mM/L, about 10 mM/L to about 100 mM/L, about 20 mM/L to about 30 mM/L, about 20 mM/L to about 40 mM/L, about 20 mM/L to about 50 mM/L, about 20 mM/L to about 60 mM/L, about 20 mM/L to about 70 mM/L, about 20 mM/L to about 80 mM/L, about 20 mM/L to about 90 mM/L, about 20 mM/L to about 100 mM/L, about 30 mM/L to about 40 mM/L, about 30 mM/L to about 50 mM/L, about 30 mM/L to about 60 mM/L, about 30 mM/L to about 70 mM/L, about 30 mM/L to about 80 mM/L, about 30 mM/L to about 90 mM/L, about 30 mM/L to about 100 mM/L, about 40 mM/L to about 50 mM/L, about 40 mM/L to about 60 mM/L, about 40 mM/L to about 70 mM/L, about 40 mM/L to about 80 mM/L, about 40 mM/L to about 90 mM/L, about 40 mM/L to about 100 mM/L, about 50 mM/L to about 60 mM/L, about 50 mM/L to about 70 mM/L, about 50 mM/L to about 80 mM/L, about 50 mM/L to about 90 mM/L, about 50 mM/L to about 100 mM/L, about 60 mM/L to about 70 mM/L, about 60 mM/L to about 80 mM/L, about 60 mM/L to about 90 mM/L, about 60 mM/L to about 100 mM/L, about 70 mM/L to about 80 mM/L, about 70 mM/L to about 90 mM/L, about 70 mM/L to about 100 mM/L, about 80 mM/L to about 90 mM/L, about 80 mM/L to about 100 mM/L, or about 90 mM/L to about 100 mM/L.

As used herein, hyaluronic acid (HA) can refer to any of its hyaluronate salts, and includes, but is not limited to, sodium hyaluronate (NaHA), potassium hyaluronate, magnesium hyaluronate, calcium hyaluronate, and combinations thereof.

Hyaluronic acid (HA) polymers are a major component of the extra-cellular matrix present throughout the human body and widely distributed in many tissues, including connective, epithelial, and neural tissues. Abundant in the different layers of the skin, HA polymers have multiple functions, including, without limitation, enhancing water retention, resisting hydrostatic stresses, ensuring good hydration, increasing soft tissue volume, assisting in the organization of the extracellular matrix, acting as a filler material in soft tissue, and participating in tissue repair mechanisms. However, the amount and quality of HA polymers and other matrix polymers present in the skin, such as collagen and elastin, decrease with age. This HA loss can result in various skin conditions such as, e.g., imperfects, defects, diseases and/or disorders, and the like, including wrinkling, hollowness, loss of moisture, drying, and other undesirable conditions that contribute to the appearance of aging.

A hyaluronic acid (HA) polymer refers to an anionic, non-sulfated glycosaminoglycan polymer, and its salts thereof, comprising disaccharide units, which themselves include D-glucuronic acid and D-N-acetylglucosamine monomers, linked together via alternating β-1,4 and β-1,3 glycosidic bonds and pharmaceutically acceptable salts thereof. Also known as a “hyaluronate polymer, or a “hyaluronan polymer”, a HA polymer can be purified from animal and non-animal sources and can range in size from about 5,000 Da to about 20,000,000 Da. It is non-immunogenic and can be chemically modified in numerous fashions. HA polymers may be anionic at pH ranges around or above the pKa of its carboxylic acid groups. Unless clearly indicated otherwise, reference to HA polymers include its fully protonated, or nonionic form, as well as any anionic forms and salts. Non-limiting examples of pharmaceutically acceptable salts of a HA polymer disclosed herein include sodium HA polymers, potassium HA polymers, lithium HA polymers, magnesium HA polymers, calcium HA polymers, and combinations thereof.

An HA may have any suitable molecular weight, such as an average molecular weight of about 5,000 Da to about 20,000,000 Da; about 300,000 Da to about 800,000 Da; or about 2,000,000 Da to about 5,000,000 Da.

A HA polymer disclosed herein comprises can comprise high molecular weight HA polymers and low molecular weight HA polymers. A high molecular weight HA polymer disclosed herein refers to a HA polymer having a mean molecular weight of at least 1.0 MDa. In some embodiments, a high molecular weight HA polymer can have a mean molecular weight of, e.g., about 1,000,000 Da, about 1,500,000 Da, about 2,000,000 Da, about 2,500,000 Da, about 3,000,000 Da, about 3,500,000 Da, about 4,000,000 Da, about 4,500,000 Da, about 5,000,000 Da, about 6,000,000 Da, about 7,000,000 Da, about 8,000,000 Da, about 9,000,000 Da, or about 10,000,000 Da. In some embodiments, a high molecular weight HA polymer can have a mean molecular weight of, e.g., at least 1,000,000 Da, at least 1,500,000 Da, at least 2,000,000 Da, at least 2,500,000 Da, at least 3,000,000 Da, at least 3,500,000 Da, at least 4,000,000 Da, at least 4,500,000 Da, or at least 5,000,000 Da, at least 6,000,000 Da, at least 7,000,000 Da, at least 8,000,000 Da, at least 9,000,000 Da, or at least 10,000,000 Da. In some embodiments, a high molecular weight HA polymer can have a mean molecular weight of, e.g., about 1,000,000 Da to about 2,000,000 Da, about 1,000,000 Da to about 3,000,000 Da, about 1,000,000 Da to about 4,000,000 Da, about 1,000,000 Da to about 5,000,000 Da, about 1,000,000 Da to about 6,000,000 Da, about 1,000,000 Da to about 7,000,000 Da, about 1,000,000 Da to about 8,000,000 Da, about 1,000,000 Da to about 9,000,000 Da, about 1,000,000 Da to about 10,000,000 Da, about 2,000,000 Da to about 3,000,000 Da, about 2,000,000 Da to about 4,000,000 Da, about 2,000,000 Da to about 5,000,000 Da, about 2,000,000 Da to about 6,000,000 Da, about 2,000,000 Da to about 7,000,000 Da, about 2,000,000 Da to about 8,000,000 Da, about 2,000,000 Da to about 9,000,000 Da, about 2,000,000 Da to about 10,000,000 Da, about 3,000,000 Da to about 4,000,000 Da, about 3,000,000 Da to about 5,000,000 Da, about 3,000,000 Da to about 6,000,000 Da, about 3,000,000 Da to about 7,000,000 Da, about 3,000,000 Da to about 8,000,000 Da, about 3,000,000 Da to about 9,000,000 Da, about 3,000,000 Da to about 10,000,000 Da, about 4,000,000 Da to about 5,000,000 Da, about 4,000,000 Da to about 6,000,000 Da, about 4,000,000 Da to about 7,000,000 Da, about 4,000,000 Da to about 8,000,000 Da, about 4,000,000 Da to about 9,000,000 Da, about 4,000,000 Da to about 10,000,000 Da, about 5,000,000 Da to about 6,000,000 Da, about 5,000,000 Da to about 7,000,000 Da, about 5,000,000 Da to about 8,000,000 Da, about 5,000,000 Da to about 9,000,000 Da, or about 5,000,000 Da to about 10,000,000 Da.

A low molecular weight HA polymer disclosed herein refers to a HA polymer having a mean molecular weight of less than 1.0 MDa. In some embodiments, a low molecular weight HA polymer can have a mean molecular weight of, e.g., about 100,000 Da, about 200,000 Da, about 300,000 Da, about 400,000 Da, about 500,000 Da, about 600,000 Da, about 700,000 Da, about 800,000 Da, about 900,000 Da, or about 950,000 Da. In some embodiments, a low molecular weight HA polymer can have a mean molecular weight of, e.g., at most 100,000 Da, at most 200,000 Da, at most 300,000 Da, at most 400,000 Da, at most 500,000 Da, at most 600,000 Da, at most 700,000 Da, at most 800,000 Da, at most 900,000 Da, or at most 950,000 Da. In some embodiments, a low molecular weight HA polymer can have a mean molecular weight of, e.g., about 100,000 Da to about 200,000 Da, about 100,000 Da to about 300,000 Da, about 100,000 Da to about 400,000 Da, about 100,000 Da to about 500,000 Da, about 100,000 Da to about 600,000 Da, about 100,000 Da to about 700,000 Da, about 100,000 Da to about 800,000 Da, about 100,000 Da to about 900,000 Da, about 100,000 Da to about 950,000 Da, about 200,000 Da to about 300,000 Da, about 200,000 Da to about 400,000 Da, about 200,000 Da to about 500,000 Da, about 200,000 Da to about 600,000 Da, about 200,000 Da to about 700,000 Da, about 200,000 Da to about 800,000 Da, about 200,000 Da to about 900,000 Da, about 200,000 Da to about 950,000 Da, about 300,000 Da to about 400,000 Da, about 300,000 Da to about 500,000 Da, about 300,000 Da to about 600,000 Da, about 300,000 Da to about 700,000 Da, about 300,000 Da to about 800,000 Da, about 300,000 Da to about 900,000 Da, about 300,000 Da to about 950,000 Da, about 400,000 Da to about 500,000 Da, about 400,000 Da to about 600,000 Da, about 400,000 Da to about 700,000 Da, about 400,000 Da to about 800,000 Da, about 400,000 Da to about 900,000 Da, about 400,000 Da to about 950,000 Da, about 500,000 Da to about 600,000 Da, about 500,000 Da to about 700,000 Da, about 500,000 Da to about 800,000 Da, about 500,000 Da to about 900,000 Da, about 500,000 Da to about 950,000 Da, about 600,000 Da to about 700,000 Da, about 600,000 Da to about 800,000 Da, about 600,000 Da to about 900,000 Da, about 600,000 Da to about 950,000 Da, about 700,000 Da to about 800,000 Da, about 700,000 Da to about 900,000 Da, about 700,000 Da to about 950,000 Da, about 800,000 Da to about 900,000 Da, about 800,000 Da to about 950,000 Da, or about 900,000 Da to about 950,000 Da.

A composition disclosed herein may comprise a low molecular weight HA polymer fraction and a high molecular weight HA polymer fraction. A high molecular weight HA polymer fraction includes HA polymers having a mean molecular weight of about 1,000,000 Da or greater, such as about 1,500,000 Da, about 2,000,000 Da, about 2,500,000 Da, about 3,000,000 Da, about 3,500,000 Da, about 4,000,000 Da, about 4,500,000 Da, or about 5,000,000 Da. A low molecular weight HA polymer fraction includes HA polymers having a mean molecular weight of less than about 1,000,000 Da, such as about 200,000 Da, about 300,000 Da, about 400,000 Da, about 500,000 Da, about 600,000 Da, about 700,000 Da, of about 800,000 Da, or about 900,000 Da.

A composition disclosed herein comprising a low molecular weight HA polymer fraction and a high molecular weight HA polymer fraction, any suitable ratio of high molecular weight HA polymers to low molecular weight HA polymers may be used. In some embodiments the weight ratio of high molecular weight HA polymers to low molecular weight HA polymers may be about 20, about 15, about 10, about 5, about 1, about 0.07, about 0.05, about 0.2, or about 0.1 to 1. A weight ratio is the quotient (weight high molecular weight HA polymer)/(low molecular weight HA polymer). For example a composition having 20 g of high molecular weight HA polymer and 1 g low molecular weight HA polymer has a weight ratio of high molecular weight HA to low molecular weight HA of 20.

A composition disclosed herein may comprise crosslinked HA polymers. As used herein, the term “crosslinked” refers to the intermolecular bonds joining two or more individual HA polymers, or monomer chains, either directly or by a linking moiety. In order to be effective in optimal duration as a dermal filler, HA polymers are usually chemically crosslinked, since non-crosslinked HA has a short persistence time in vivo. Crosslinked HA polymers can create a more stable structure which can increase the viscosity of an aqueous composition, and which may result in the formation of a hydrogel. As such, a crosslinked HA polymer has at least one intermolecular bond joining at least one individual HA polymer to another HA polymer.

HA polymers may be crosslinked using crosslinking agents. A disclosed crosslinking agent may be any agent known to be suitable for crosslinking polysaccharides and their derivatives via their hydroxyl groups. In some embodiments, a crosslinking agent used to crosslink HA polymers include dialdehyde or disulfide crosslinking agents including, without limitation, multifunctional PEG-based crosslinking agents, divinyl sulfones, diglycidyl ethers, bis-epoxides, and biscarbodiimides. Non-limiting examples of crosslinking agents include multifunctional PEG-based crosslinking agents like pentaerythritol tetraglycidyl ether (PETGE), divinyl sulfone (DVS), 1,4-butanediol diglycidyl ether (BDDE), 1,2-bis(2,3-epoxypropoxy)ethylene (EGDGE), 1,2,7,8-diepoxyoctane (DEO), (phenylenebis-(ethyl)-carbodiimide and 1,6 hexamethylenebis (ethylcarbodiimide)), adipic dihydrazide (ADH), bis(sulfosuccinimidyl)suberate (BS), hexamethylenediamine (HMDA), 1-(2,3-epoxypropyl)-2,3-epoxycyclohexane, or combinations thereof. Other useful cross-linking agents are disclosed in U.S. Pat. No. 8,318,695, the content of which is hereby incorporated by reference in its entirety. Chemical crosslinking methods include Michael addition, thiol-ene coupling, free radical polymerization, carbodiimide chemistry (e.g., 1-ethyl-3-(3-dimethylaminopropyl) carbodiimide (EDC)) using a di- or polyamine as a crosslinker, and epoxy chemistry using 1,4-butanediol diglycidyl ether (BDDE) as a crosslinker. Other non-limiting methods of crosslinking HA polymers are described in, e.g., U.S. Pat. Nos. 6,921,819, 7,741,476, 8,338,388, 8,357,795, 8,450,475, 8,563,532, 9,062,130, 10,080,767, 10,653,716, the content of each of which is hereby incorporated by reference in its entirety.

A crosslinked HA polymer can be defined by its degree of crosslinking. The degree of crosslinking of a HA polymer refers to the intermolecular links joining individual HA polymers into a permanent structure. The degree of crosslinking can be quantified as the percentage of crosslinking agent to monomeric units (i.e. the disaccharide monomer unit) that are bound within the crosslinked portion of the HA polymers. Thus, crosslinked HA polymers with a 4% degree of crosslinking means that on average there are four crosslinking molecules for every 100 monomeric units of the crosslinked portion of the HA polymers. Every other parameter being equal, the greater the degree of crosslinking, the harder the hydrogel becomes.

In some embodiments, crosslinked HA polymers have a degree of crosslinking necessary and sufficient to form a hydrogel. In aspects of these embodiments, crosslinked HA polymers have a degree of crosslinking of, e.g., about 1%, about 2%, about 3%, about 4%, about 5%, about 6%, about 7%, about 8%, about 9%, about 10%, about 11%, about 12%, about 13%, about 14%, or about 15%. In other aspects of these embodiments, crosslinked HA polymers have a degree of crosslinking of, e.g., at least 1%, at least 2%, at least 3%, at least 4%, at least 5%, at least 6%, at least 7%, at least 8%, at least 9%, at least 10%, at least 11%, at least 12%, at least 13%, at least 14%, or at least 15%. In yet other aspects of these embodiments, crosslinked HA polymers have a degree of crosslinking of, e.g., at most 1%, at most 2%, at most 3%, at most 4%, at most 5%, at most 6%, at most 7%, at most 8%, at most 9%, at most 10%, at most 11%, at most 12%, at most 13%, at most 14%, or at most 15%. In still other aspects of these embodiments, crosslinked HA polymers have a degree of crosslinking of, e.g., about 1% to about 3%, about 1% to about 5%, about 1% to about 7%, about 1% to about 10%, about 1% to about 15%, about 3% to about 15%, about 5% to about 15%, about 7% to about 15%, about 9% to about 15%, about 10% to about 15%, about 11% to about 15%, about 2% to about 11%, about 3% to about 11%, about 4% to about 11%, about 5% to about 11%, about 6% to about 11%, about 7% to about 11%, about 8% to about 11%, about 9% to about 11%, about 3% to about 10%, about 4% to about 10%, about 5% to about 10%, about 6% to about 10%, about 7% to about 10%, about 8% to about 10%, about 4% to about 9%, about 5% to about 9%, about 6% to about 9%, about 7% to about 9%, about 5% to about 8%, or about 6% to about 8%.

A composition disclosed herein can comprise crosslinked HA polymers of low molecular weight, crosslinked HA polymers of high molecular weight, or crosslinked HA polymers of both low and high molecular weight.

In some embodiments, a composition disclosed herein comprises crosslinked HA polymers of low molecular weight. In aspects of these embodiments, a composition disclosed herein comprises crosslinked HA polymers of low molecular weight having a mean molecular weight of, e.g., about 100,000 Da, about 200,000 Da, about 300,000 Da, about 400,000 Da, about 500,000 Da, about 600,000 Da, about 700,000 Da, about 800,000 Da, or about 900,000 Da. In yet other aspects of these embodiments, a composition disclosed herein comprises crosslinked HA polymers of low molecular weight having a mean molecular weight of, e.g., at most 100,000 Da, at most 200,000 Da, at most 300,000 Da, at most 400,000 Da, at most 500,000 Da, at most 600,000 Da, at most 700,000 Da, at most 800,000 Da, at most 900,000 Da, or at most 950,000. In still other aspects of these embodiments, a composition disclosed herein comprises crosslinked HA polymers of low molecular weight having a mean molecular weight of, e.g., about 100,000 Da to about 500,000 Da, about 200,000 Da to about 500,000 Da, about 300,000 Da to about 500,000 Da, about 400,000 Da to about 500,000 Da, about 500,000 Da to about 950,000 Da, about 600,000 Da to about 950,000 Da, about 700,000 Da to about 950,000 Da, about 800,000 Da to about 950,000 Da, about 300,000 Da to about 600,000 Da, about 300,000 Da to about 700,000 Da, about 300,000 Da to about 800,000 Da, or about 400,000 Da to about 700,000 Da.

In some embodiments, a composition disclosed herein comprises crosslinked HA polymers of high molecular weight. In aspects of these embodiments, a composition disclosed herein comprises crosslinked HA polymers of high molecular weight having a mean molecular weight of, e.g., about 1,000,000 Da, about 1,500,000 Da, about 2,000,000 Da, about 2,500,000 Da, about 3,000,000 Da, about 3,500,000 Da, about 4,000,000 Da, about 4,500,000 Da, or about 5,000,000 Da. In yet other aspects of these embodiments, a composition disclosed herein comprises crosslinked HA polymers of high molecular weight having a mean molecular weight of, e.g., at least 1,000,000 Da, at least 1,500,000 Da, at least 2,000,000 Da, at least 2,500,000 Da, at least 3,000,000 Da, at least 3,500,000 Da, at least 4,000,000 Da, at least 4,500,000 Da, or at least 5,000,000 Da. In still other aspects of these embodiments, a composition disclosed herein comprises crosslinked HA polymers of high molecular weight having a mean molecular weight of, e.g., about 1,000,000 Da to about 5,000,000 Da, about 1,500,000 Da to about 5,000,000 Da, about 2,000,000 Da to about 5,000,000 Da, about 2,500,000 Da to about 5,000,000 Da, about 2,000,000 Da to about 3,000,000 Da, about 2,500,000 Da to about 3,500,000 Da, or about 2,000,000 Da to about 4,000,000 Da.

In some embodiments, a composition disclosed herein comprises crosslinked HA polymers where the crosslinked HA polymers comprise a combination of both high molecular weight HA polymers and low molecular weight HA polymers. In aspects of these embodiments, a composition disclosed herein comprises a crosslinked HA polymers where the crosslinked HA polymers comprise a combination of both high molecular weight HA polymers and low molecular weight HA polymers in a ratio of about 20:1, about 15:1, about 10:1, about 5:1, about 1:1, about 1:5 about 1:10, about 1:15, or about 1:20.

In some embodiments, a composition disclosed herein comprises crosslinked HA polymer in an amount necessary and sufficient to improve a skin condition as disclosed herein. In aspect of these embodiments, a composition disclosed herein comprises crosslinked HA polymers in an amount of, e.g., about 1%, about 2%, about 3%, about 4%, about 5%, about 6%, about 7%, about 8%, or about 9%, or about 10% by weight of the composition. In other aspects of these embodiments, a composition disclosed herein comprises crosslinked HA polymers in an amount of, e.g., at least 1%, at least 2%, at least 3%, at least 4%, at least 5%, at least 6%, at least 7%, at least 8%, at least 9%, or at least 10% by weight of the composition. In yet other aspects of these embodiments, a composition disclosed herein comprises crosslinked HA polymers in an amount of, e.g., at most 1%, at most 2%, at most 3%, at most 4%, at most 5%, at most 6%, at most 7%, at most 8%, at most 9%, or at most 10% by weight of the composition. In still other aspects of these embodiments, a composition disclosed herein comprises crosslinked HA polymers in an amount of, e.g., about 1% to about 10%, about 2% to about 10%, about 3% to about 10%, about 4% to about 10%, about 5% to about 10%, about 6% to about 10%, about 7% to about 10%, about 1% to about 9%, about 2% to about 9%, about 3% to about 9%, about 4% to about 9%, about 5% to about 9%, about 6% to about 9%, or about 7% to about 9%, about 1% to about 8%, about 2% to about 8%, about 3% to about 8%, about 4% to about 8%, about 5% to about 8%, about 6% to about 8%, about 1% to about 7%, about 2% to about 7%, about 3% to about 7%, about 4% to about 7%, about 5% to about 7%, about 1% to about 6%, about 2% to about 6%, about 3% to about 6%, about 4% to about 6%, about 1% to about 5%, about 2% to about 5%, about 3% to about 5%, about 1% to about 4%, about 2% to about 4%, about 1% to about 3%, by weight of the composition.

In aspect of these embodiments, a composition disclosed herein comprises crosslinked HA polymers in an amount of, e.g., about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 13.5 mg/mL, about 14 mg/mL, about 15 mg/mL, about 16 mg/mL, about 17 mg/mL, about 18 mg/mL, about 19 mg/mL, or about 20 mg/mL. In other aspects of these embodiments, a composition disclosed herein comprises crosslinked HA polymers in an amount of, e.g., at least 1 mg/mL, at least 2 mg/mL, at least 3 mg/mL, at least 4 mg/mL, at least 5 mg/mL, at least 10 mg/mL, at least 15 mg/mL, at least 20 mg/mL, or at least 25 mg/mL. In yet other aspects of these embodiments, a composition disclosed herein comprises crosslinked HA polymers in an amount of, e.g., at most 1 mg/mL, at most 2 mg/mL, at most 3 mg/mL, at most 4 mg/mL, at most 5 mg/mL, at most 10 mg/mL, at most 15 mg/mL, at most 20 mg/mL, or at most 25 mg/mL. In still other aspects of these embodiments, a composition disclosed herein comprises crosslinked HA polymers in an amount of, e.g., about 7.5 mg/mL to about 19.5 mg/mL, about 8.5 mg/mL to about 18.5 mg/mL, about 9.5 mg/mL to about 17.5 mg/mL, about 10.5 mg/mL to about 16.5 mg/mL, about 11.5 mg/mL to about 15.5 mg/mL, or about 12.5 mg/mL to about 14.5 mg/mL.

In other aspects of these embodiments, a composition disclosed herein comprises crosslinked HA polymers in an amount of, e.g., about 15 mg/mL, about 20 mg/mL, about 25 mg/mL, about 30 mg/mL, about 35 mg/mL, or about 40 mg/mL. In other aspects of these embodiments, a composition disclosed herein comprises crosslinked HA polymers in an amount of, e.g., at least 15 mg/mL, at least 20 mg/mL, at least 25 mg/mL, at least 30 mg/mL, at least 35 mg/mL, or at least 40 mg/mL. In yet other aspects of these embodiments, a composition disclosed herein comprises crosslinked HA polymers in an amount of, e.g., at most 15 mg/mL, at most 20 mg/mL, at most 25 mg/mL, at most 30 mg/mL, at most 35 mg/mL, or at most 40 mg/mL. In still other aspects of these embodiments, a composition disclosed herein comprises crosslinked HA polymers in an amount of, e.g., about 15 mg/mL to about 20 mg/mL, about 15 mg/mL to about 25 mg/mL, about 15 mg/mL to about 30 mg/mL, about 15 mg/mL to about 35 mg/mL, about 15 mg/mL to about 40 mg/mL, about 20 mg/mL to about 25 mg/mL, about 20 mg/mL to about 30 mg/mL, about 20 mg/mL to about 35 mg/mL, about 20 mg/mL to about 40 mg/mL, about 25 mg/mL to about 30 mg/mL, about 25 mg/mL to about 35 mg/mL, about 25 mg/mL to about 40 mg/mL, about 30 mg/mL to about 35 mg/mL, about 30 mg/mL to about 40 mg/mL, or about 35 mg/mL to about 40 mg/mL.

A composition disclosed herein may comprise uncrosslinked HA polymers. As used herein, the term “uncrosslinked” refers to a lack of intermolecular bonds joining the individual HA polymers, or monomer chains. As such, an uncrosslinked HA polymer is not linked to any other HA polymer by an intermolecular bond. Uncrosslinked HA polymers are water soluble and generally remain fluid in nature. As such, uncrosslinked HA polymers can be added to a composition disclosed herein to improve the rheological properties of a composition disclosed herein so as to improve treatment of a skin condition as well as a lubricant to facilitate the extrusion process of the composition through a fine needle.

A composition disclosed herein can comprise uncrosslinked HA polymers of low molecular weight, uncrosslinked HA polymers of high molecular weight, or uncrosslinked HA polymers of both low and high molecular weight.

In some embodiments, a composition disclosed herein comprises uncrosslinked HA polymers of low molecular weight. In aspects of these embodiments, a composition disclosed herein comprises uncrosslinked HA polymers of low molecular weight having a mean molecular weight of, e.g., about 100,000 Da, about 200,000 Da, about 300,000 Da, about 400,000 Da, about 500,000 Da, about 600,000 Da, about 700,000 Da, about 800,000 Da, or about 900,000 Da. In yet other aspects of these embodiments, a composition disclosed herein comprises uncrosslinked HA polymers of low molecular weight having a mean molecular weight of, e.g., at most 100,000 Da, at most 200,000 Da, at most 300,000 Da, at most 400,000 Da, at most 500,000 Da, at most 600,000 Da, at most 700,000 Da, at most 800,000 Da, at most 900,000 Da, or at most 950,000. In still other aspects of these embodiments, a composition disclosed herein comprises uncrosslinked HA polymers of low molecular weight having a mean molecular weight of, e.g., about 100,000 Da to about 500,000 Da, about 200,000 Da to about 500,000 Da, about 300,000 Da to about 500,000 Da, about 400,000 Da to about 500,000 Da, about 500,000 Da to about 950,000 Da, about 600,000 Da to about 950,000 Da, about 700,000 Da to about 950,000 Da, about 800,000 Da to about 950,000 Da, about 300,000 Da to about 600,000 Da, about 300,000 Da to about 700,000 Da, about 300,000 Da to about 800,000 Da, or about 400,000 Da to about 700,000 Da.

In some embodiments, a composition disclosed herein comprises uncrosslinked HA polymers of high molecular weight. In aspects of these embodiments, a composition disclosed herein comprises uncrosslinked HA polymers of high molecular weight having a mean molecular weight of, e.g., about 1,000,000 Da, about 1,500,000 Da, about 2,000,000 Da, about 2,500,000 Da, about 3,000,000 Da, about 3,500,000 Da, about 4,000,000 Da, about 4,500,000 Da, or about 5,000,000 Da. In other aspects of these embodiments, a composition disclosed herein comprises uncrosslinked HA polymers of high molecular weight having a mean molecular weight of, e.g., at least 1,000,000 Da, at least 1,500,000 Da, at least 2,000,000 Da, at least 2,500,000 Da, at least 3,000,000 Da, at least 3,500,000 Da, at least 4,000,000 Da, at least 4,500,000 Da, or at least 5,000,000 Da. In yet other aspects of these embodiments, a composition disclosed herein comprises uncrosslinked HA polymers of high molecular weight having a mean molecular weight of, e.g., about 1,000,000 Da to about 5,000,000 Da, about 1,500,000 Da to about 5,000,000 Da, about 2,000,000 Da to about 5,000,000 Da, about 2,500,000 Da to about 5,000,000 Da, about 2,000,000 Da to about 3,000,000 Da, about 2,500,000 Da to about 3,500,000 Da, or about 2,000,000 Da to about 4,000,000 Da. In still other aspects these embodiments, a composition disclosed herein comprises uncrosslinked HA polymers of high molecular weight having a mean molecular weight of, e.g., greater than 2,000,000 Da and less than about 3,000,000 Da, greater than 2,000,000 Da and less than about 3,500,000 Da, greater than 2,000,000 Da and less than about 4,000,000 Da, greater than 2,000,000 Da and less than about 4,500,000 Da, greater than 2,000,000 Da and less than about 5,000,000 Da.

In some embodiment, a composition disclosed herein comprises uncrosslinked HA polymers where the uncrosslinked HA comprises a combination of both high molecular weight HA polymers and low molecular weight HA polymers. In aspects of these embodiments, a composition disclosed herein comprises uncrosslinked hyaluronan polymers where the uncrosslinked hyaluronan polymers comprises a combination of both high molecular weight hyaluronan polymers and low molecular weight hyaluronan polymers in a ratio of about 20:1, about 15:1, about 10:1, about 5:1, about 1:1, about 1:5 about 1:10, about 1:15, or about 1:20.

In some embodiments, a composition disclosed herein comprises uncrosslinked HA polymers in an amount necessary and sufficient to improve the rheological properties of a composition. In aspect of these embodiments, a composition disclosed herein comprises uncrosslinked HA polymers in an amount of, e.g., about 0.1%, about 0.5%, about 1%, about 2%, about 3%, about 4%, about 5%, about 6%, about 7%, about 8%, or about 9%, or about 10% by weight of the composition. In other aspects of these embodiments, a composition disclosed herein comprises uncrosslinked HA polymers in an amount of, e.g., at most 0.1%, at most 0.5%, at most 1%, at most 2%, at most 3%, at most 4%, at most 5%, at most 6%, at most 7%, at most 8%, at most 9%, or at most 10% by weight of the composition. In yet other aspects of these embodiments, a composition disclosed herein comprises uncrosslinked HA polymers in an amount of, e.g., about 0.1% to about 1%, about 0.5% to about 1%, about 0.1% to about 2%, about 0.5% to about 2%, about 1% to about 2%, about 0.1% to about 3%, about 0.5% to about 3%, about 1% to about 3%, about 0.1% to about 5%, about 0.5% to about 5%, about 1% to about 5%, about 3% to about 5%, about 0.1% to about 10%, about 0.5% to about 10%, about 1% to about 10%, about 3% to about 10%, or about 5% to about 10% by weight of the composition.

In aspects of these embodiments, a composition disclosed herein comprises uncrosslinked HA polymers in an amount of, e.g., about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 13.5 mg/mL, about 14 mg/mL, about 15 mg/mL, about 16 mg/mL, about 17 mg/mL, about 18 mg/mL, about 19 mg/mL, about 20 mg/mL, about 40 mg/mL, or about 60 mg/mL. In other aspects of these embodiments, a composition disclosed herein comprises uncrosslinked HA polymers in an amount of, e.g., at least 1 mg/mL, at least 2 mg/mL, at least 3 mg/mL, at least 4 mg/mL, at least 5 mg/mL, at least 10 mg/mL, at least 15 mg/mL, at least 20 mg/mL, at least 25 mg/mL at least 35 mg/mL, or at least 40 mg/mL. In yet other aspects of these embodiments, a composition disclosed herein comprises uncrosslinked HA polymers in an amount of, e.g., at most 1 mg/mL, at most 2 mg/mL, at most 3 mg/mL, at most 4 mg/mL, at most 5 mg/mL, at most 10 mg/mL, at most 15 mg/mL, at most 20 mg/mL, or at most 25 mg/mL. In still other aspects of these embodiments, a composition disclosed herein comprises uncrosslinked HA polymers in an amount of, e.g., about 1 mg/mL to about 60 mg/mL, about 10 mg/mL to about 40 mg/mL, about 7.5 mg/mL to about 19.5 mg/mL, about 8.5 mg/mL to about 18.5 mg/mL, about 9.5 mg/mL to about 17.5 mg/mL, about 10.5 mg/mL to about 16.5 mg/mL, about 11.5 mg/mL to about 15.5 mg/mL, or about 12.5 mg/mL to about 14.5 mg/mL.

In some embodiments, a composition disclosed herein comprises a mixture of crosslinked HA polymers and uncrosslinked HA polymer. In aspects of these embodiments, a composition disclosed herein comprises, e.g., about 10%, about 20%, about 30%, about 40%, about 50%, about 60%, about 70%, about 80%, about 90%, about 95%, or about 100% of crosslinked HA polymers by weight relative to the total amount of HA polymers present in the composition. In other aspects of these embodiments, a composition disclosed herein comprises, e.g., at least 10%, at least 20%, at least 30%, at least 40%, at least 50%, at least 60%, at least 70%, at least 80%, at least 90%, at least 95%, or at least 99% of crosslinked HA polymers by weight relative to the total amount of HA polymers present in the composition. In yet other aspects of these embodiments, a composition disclosed herein comprises, e.g., at most 10%, at most 20%, at most 30%, at most 40%, at most 50%, at most 60%, at most 70%, at most 80%, at most 90%, at most 95%, or at most 99% of crosslinked HA polymers by weight relative to the total amount of HA polymers present in the composition.

In yet other aspects of these embodiments, a composition disclosed herein comprises, e.g., about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 80% to about 100% of crosslinked HA polymers by weight relative to the total amount of HA polymers present in the composition.

A composition disclosed herein can comprises a ratio of crosslinked HA polymers and uncrosslinked HA polymers. This ratio of crosslinked and uncrosslinked HA polymer is also known as the gel:fluid ratio. Any gel:fluid ratio is useful in making a composition disclosed herein with the proviso that such ratio produces a composition disclosed herein that improves a skin condition as disclosed herein. It will be appreciated by those of ordinary skill in the art that the selection of high and low molecular weight HA material and their relative percentages or gel:fluid ratios is dependent upon the desired characteristics, for example, extrusion force, elastic modulus, viscous modulus and phase angle expressed as the ratio of viscous modulus to elastic modulus, cohesivity, etc. of the final HA-based product. Non-limiting examples of gel:fluid ratios include 100:0, 98:2, 90:10, 75:25, 70:30, 60:40, 50:50, 40:60, 30:70, 25:75, 10:90; 2:98, and 0:100.

In aspects of this embodiment, a composition disclosed herein comprises crosslinked HA polymers and uncrosslinked HA polymers having a gel:fluid ratio of, e.g., about 0:100, about 1:99, about 2:98, about 3:97, about 4:96, about 5:95, about 6:94, about 7:93, about 8:92, about 9:91, or about 10:90. In other aspects of this embodiment, a composition disclosed herein comprises crosslinked HA polymers and uncrosslinked HA polymers having a gel:fluid ratio of, e.g., at most 1:99, at most 2:98, at most 3:97, at most 4:96, at most 5:95, at most 6:94, at most 7:93, at most 8:92, at most 9:91, or at most 10:90. In yet other aspects of this embodiment, a composition disclosed herein comprises crosslinked HA polymers and uncrosslinked HA polymers having a gel:fluid ratio of, e.g., about 0.001:100 to about 1:100, about 0.005:100 to about 1:100, about 0.01:100 to about 1:100, about 0.05:100 to about 1:100, about 0.1:100 to about 1:100, about 0.5:100 to about 1:100, about 0:100 to about 3:97, about 0:100 to about 5:95, or about 0:100 to about 10:90.

In other aspects of this embodiment, a composition disclosed herein comprises crosslinked HA polymers and uncrosslinked HA polymers having a gel:fluid ratio of, e.g., about 15:85, about 20:80, about 25:75, about 30:70, about 35:65, about 40:60, about 45:55, about 50:50, about 55:45, about 60:40, about 65:35, about 70:30, about 75:25, about 80:20, about 85:15, about 90:10, about 95:5, about 98:2, or about 100:0. In yet other aspects of this embodiment, a composition disclosed herein comprises crosslinked HA polymers and uncrosslinked HA polymers having a gel:fluid ratio of, e.g., at most 15:85, at most 20:80, at most 25:75, at most 30:70, at most 35:65, at most 40:60, at most 45:55, at most 50:50, at most 55:45, at most 60:40, at most 65:35, at most 70:30, at most 75:25, at most 80:20, at most 85:15, at most 90:10, at most 95:5, at most 98:2, or at most 100:0. In still other aspects of this embodiment, a composition disclosed herein comprises crosslinked HA polymers and uncrosslinked HA polymers having a gel:fluid ratio of, e.g., about 10:90 to about 70:30, about 15:85 to about 70:30, about 10:90 to about 55:45, about 80:20 to about 95:5, about 90:10 to about 100:0, about 75:25 to about 100:0, or about 60:40 to about 100:0.

A monoterpene compound-HA polymer conjugate is a molecule having a monoterpene compound disclosed herein covalently bound via a chemical linking group to a HA polymer disclosed herein. A monoterpene compound-HA polymer conjugate has improved or increased dermal penetration activity relative to HA polymer not conjugated to a monoterpene compound disclosed herein. In addition, a monoterpene compound-HA polymer conjugate enhances an activity of a monoterpene compound thus conjugated by, e.g., enhancing the stability of the monoterpene compound.

In some embodiments, the molar ratio of monoterpene compound disclosed herein to a HA polymer disclosed herein in such conjugates may be e.g., 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, or any other suitable molar ratios. In some embodiments, the molar ratio of monoterpene compound disclosed herein to a HA polymer disclosed herein in such conjugates may be e.g., 2:1, 3:1, 4:1, 5:1, 6:1, 7:1, 8:1, 9:1, 10:1, or any other suitable molar ratios. The monoterpene compound and a HA polymer may be covalently linked, through carbamate, ester, ether bonds, or any other suitable chemical functional groups. When the monoterpene compound and a HA polymer are conjugated through a carbamate bond, a HA polymer may be any agent bearing at least one carboxylic acid functional group, or at least one amine functional group. In a specific example, a perillyl alcohol-HA polymer conjugate is perillyl alcohol, or derivative thereof, covalently bound via a chemical linking group to a HA polymer. Methods of crossing polyhydric alcohols to HA polymers are described in, e.g., U.S. Pat. No. 4,957,744, the content of which is hereby incorporated by reference in its entirety.

In some embodiments, a composition disclosed herein comprises one or more monoterpene compounds disclosed herein and HA polymers disclosed herein as an admixture. In aspects of these embodiments, a composition disclosed herein comprises 10 mM to 100 mM of one or more monoterpene compounds disclosed herein and 0.5% to 10% of HA polymers disclosed herein as an admixture. In other aspects of these embodiments, a composition disclosed herein comprises 20 mM to 80 mM of one or more monoterpene compounds disclosed herein and 1% to 8% of HA polymers disclosed herein as an admixture. In other aspects of these embodiments, a composition disclosed herein comprises 30 mM to 70 mM of one or more monoterpene compounds disclosed herein and 2% to 6% of HA polymers disclosed herein as an admixture.

In some embodiments, a composition disclosed herein comprises a perillyl alcohol disclosed herein and HA polymers disclosed herein as an admixture. In aspects of these embodiments, a composition disclosed herein comprises 10 mM to 100 mM of a perillyl alcohol disclosed herein and 0.5% to 10% of HA polymers disclosed herein as an admixture. In other aspects of these embodiments, a composition disclosed herein comprises 20 mM to 80 mM of a perillyl alcohol disclosed herein and 1% to 8% of HA polymers disclosed herein as an admixture. In other aspects of these embodiments, a composition disclosed herein comprises 30 mM to 70 mM of a perillyl alcohol disclosed herein and 2% to 6% of HA polymers disclosed herein as an admixture.

In some embodiments, composition disclosed herein comprises one or more monoterpene compounds disclosed herein, HA polymers disclosed herein, and one or more monoterpene compound-HA polymer conjugates disclosed herein. In aspects of these embodiments, a composition disclosed herein comprises 10 mM to 100 mM of one or more monoterpene compounds disclosed herein, 0.5% to 10% of HA polymers disclosed herein, and 0.5% to 10% of one or more monoterpene compound-HA polymer conjugates disclosed herein where the amount of monoterpene compounds in the one or more monoterpene compound-HA polymer conjugates is equivalent to 10 mM to 100 mM. In other aspects of these embodiments, a composition disclosed herein comprises 20 mM to 80 mM of one or more monoterpene compounds disclosed herein, 1% to 8% HA of polymers disclosed herein, and 1% to 8% of one or more monoterpene compound-HA polymer conjugates disclosed herein where the amount of monoterpene compounds in the one or more monoterpene compound-HA polymer conjugates is equivalent to 20 mM to 80 mM. In other aspects of these embodiments, a composition disclosed herein comprises 30 mM to 70 mM of one or more monoterpene compounds disclosed herein, 2% to 6% HA of polymers disclosed herein, and 1% to 6% of one or more monoterpene compound-HA polymer conjugates disclosed herein where the amount of monoterpene compounds in the one or more monoterpene compound-HA polymer conjugates is equivalent to 30 mM to 70 mM.

In some embodiments, composition disclosed herein comprises a perillyl alcohol disclosed herein, HA polymers disclosed herein, and a perillyl alcohol-HA polymer conjugate disclosed herein. In aspects of these embodiments, a composition disclosed herein comprises 10 mM to 100 mM of a perillyl alcohol disclosed herein, 0.5% to 10% of HA polymers disclosed herein, and 0.5% to 10% of a perillyl alcohol HA polymer conjugate disclosed herein where the amount of perillyl alcohol in the perillyl alcohol-HA polymer conjugate is equivalent to 10 mM to 100 mM. In other aspects of these embodiments, a composition disclosed herein comprises 20 mM to 80 mM of a perillyl alcohol disclosed herein, 1% to 8% of HA polymers disclosed herein, and 1% to 8% of a perillyl alcohol-HA polymer conjugate disclosed herein where the amount of perillyl alcohol in the perillyl alcohol-HA polymer conjugate is equivalent to 20 mM to 80 mM. In other aspects of these embodiments, a composition disclosed herein comprises 30 mM to 70 mM a perillyl alcohol disclosed herein, 2% to 6% of HA polymers disclosed herein, and 1% to 6% of a perillyl alcohol-HA polymer conjugate disclosed herein where the amount of perillyl alcohol in the perillyl alcohol-HA polymer conjugate is equivalent to 30 mM to 70 mM.

In some embodiments, composition disclosed herein comprises one or more monoterpene compound-HA polymer conjugates disclosed herein. In aspects of these embodiments, a composition disclosed herein comprises 0.5% to 10% of one or more monoterpene compound-HA polymer conjugates disclosed herein where the amount of monoterpene compounds in the one or more monoterpene compound-HA polymer conjugates is equivalent to 10 mM to 100 mM. In other aspects of these embodiments a composition disclosed herein comprises 1% to 8% of one or more monoterpene compound-HA polymer conjugates disclosed herein where the amount of monoterpene compounds in the one or more monoterpene compound-HA polymer conjugates equivalent to 20 mM to 80 mM. In other aspects of these embodiments, a composition disclosed herein comprises 2% to 6% of one or more monoterpene compound-HA polymer conjugates disclosed herein where the amount of monoterpene compounds in the one or more monoterpene compound-HA polymer conjugates equivalent to 30 mM to 70 mM.

In some embodiments, composition disclosed herein comprises a perillyl alcohol-HA polymer conjugate disclosed herein. In aspects of these embodiments, a composition disclosed herein comprises 0.5% to 10% of a perillyl alcohol-HA polymer conjugate disclosed herein where the amount of perillyl alcohol in the perillyl alcohol-HA polymer conjugate is equivalent to 10 mM to 100 mM. In other aspects of these embodiments a composition disclosed herein comprises 1% to 8% of a perillyl alcohol-HA polymer conjugate disclosed herein where the amount of perillyl alcohol in the perillyl alcohol-HA polymer conjugate equivalent to 20 mM to 80 mM. In other aspects of these embodiments, a composition disclosed herein comprises 2% to 6% a perillyl alcohol-HA polymer conjugate disclosed herein where the amount of perillyl alcohol in the perillyl alcohol-HA polymer conjugate equivalent to 30 mM to 70 mM.

A composition disclosed herein may comprise a carrier. A carrier, also known as a vehicle, can be any material typically known in the skin care, cosmetic and medical arts that is used as a base to formulate a composition disclosed herein. A carrier has substantially no long term or permanent detrimental effect when administered and encompasses terms such as vehicle, stabilizer, diluent, additive, auxiliary, or excipient. Such a carrier generally is mixed with a monoterpene compound, HA polymers, and/or a monoterpene compound-HA polymer conjugate disclosed herein or permitted to dilute or enclose a monoterpene compound, HA polymers, and/or a monoterpene compound-HA polymer conjugate disclosed herein. It is understood that a monoterpene compound and/or HA polymers disclosed herein can be soluble or can be delivered as a suspension in the desired carrier or diluent. A carrier may be an aqueous carrier, a semi-solid carrier or a solid carrier. A carrier can also provide a skin care benefit as disclosed herein. Selection of a pharmacologically acceptable carrier can depend on the mode of administration. Except insofar as any pharmacologically acceptable carrier is incompatible with a monoterpene compound, HA polymers, and/or a monoterpene compound-HA polymer conjugate disclosed herein, its use in pharmaceutically acceptable compositions is contemplated.

A carrier includes, without limitation, water, a vegetable oil, a mineral oil, an ester oil, an ether, an alcohol, a fatty alcohol, an isoparaffin, a hydrocarbon oil, a polyol, and a wax. Non-limiting examples of an ester oil include octal palmitate, isopropyl myristate and isopropyl palmitate. Non-limiting examples of an ether includes dicapryl ether and dimethyl isosorbide. Non-limiting examples of an alcohol includes ethanol and isopropanol. Non-limiting examples of a fatty alcohol include cetyl alcohol, cetearyl alcohol, stearyl alcohol and behenyl alcohol. Non-limiting examples of an isoparaffin include isooctane, isododecane (IDD) and isohexadecane. Non-limiting examples of a hydrocarbon oil include mineral oil, petrolatum, isoeicosane and a polyolefin, including (hydrogenated) polyisobutene. Non-limiting examples of a polyol include propylene glycol, glycerin, butylene glycol, pentylene glycol, hexylene glycol, caprylyl glycol. Non-limiting examples of a wax include beeswax, carnauba, ozokerite, microcrystalline wax, polyethylene wax, and a botanical wax. Non-limiting examples of specific uses of such carriers can be found in Pharmaceutical Dosage Forms and Drug Delivery Systems (Howard C. Ansel et al., eds., Lippincott Williams & Wilkins Publishers, 7.sup.th ed. 1999); Remington: The Science and Practice of Pharmacy (Alfonso R. Gennaro ed., Lippincott, Williams & Wilkins, 20.sup.th ed. 2000); Goodman & Gilman's The Pharmacological Basis of Therapeutics (Joel G. Hardman et al., eds., McGraw-Hill Professional, 10.sup.th ed. 2001); and Handbook of Pharmaceutical Excipients (Raymond C. Rowe et al., APhA Publications, 4.sup.th edition 2003).

In one embodiment, a single carrier is present in a composition disclosed herein. In another embodiment, a plurality of carriers is present in a composition disclosed herein. In aspects of this embodiment, a composition disclosed herein comprises, e.g., one or more carriers, two or more carriers, three or more carriers, four or more carriers or five or more carriers. In other aspects of this embodiment, a composition disclosed herein comprises, e.g., only one carrier, at most two carriers, at most three carriers, at most four carriers, or at most five carriers. In yet other aspects of this embodiment, a composition disclosed herein comprises from, e.g., 1 to 2 carriers, 1 to 3 carriers, 1 to 4 carriers, 1 to 5 carriers, 2 to 3 carriers, 2 to 4 carriers, 2 to 5 carriers, 3 to 4 carriers, 3 to 5 carriers or 4 to 5 carriers.

In another embodiment, a composition disclosed herein comprises an amount of carrier that provides a desired formulative or beneficial effect to a composition disclosed herein. In aspects of this embodiment, a composition disclosed herein comprises a carrier in an amount of, e.g., at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98% or at least 99% by weight of the composition. In other aspects of this embodiment, a composition disclosed herein comprises a carrier in an amount of, e.g., at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 96%, at most 97%, at most 98% or at most 99% by weight of the composition. In yet other aspects of this embodiment, a composition disclosed herein comprises a carrier in an amount of from, e.g., about 25% to about 50%, about 25% to about 75%, about 25% to about 90%, about 25% to about 95%, about 25% to about 96%, about 25% to about 97%, about 25% to about 98%, about 25% to about 99%, about 50% to about 75%, about 50% to about 90%, about 50% to about 95%, about 50% to about 96%, about 50% to about 97%, about 50% to about 98%, about 50% to about 99%, about 75% to about 80%, about 75% to about 85%, about 75% to about 90%, about 75% to about 95%, about 75% to about 96%, about 75% to about 97%, about 75% to about 98%, about 75% to about 99%, about 80% to about 85%, about 80% to about 90%, about 80% to about 95%, about 80% to about 96%, about 80% to about 97%, about 80% to about 98%, about 80% to about 99%, about 85% to about 90%, about 85% to about 95%, about 85% to about 96%, about 85% to about 97%, about 85% to about 98%, about 85% to about 99%, about 90% to about 95%, about 90% to about 96%, about 90% to about 97%, about 90% to about 98%, about 90% to about 99%, or about 95% to about 99%, by weight of the composition.

A composition disclosed herein may optionally include, without limitation, other pharmaceutically acceptable components (or pharmaceutical components), including, without limitation, buffers, preservatives, tonicity adjusters, salts, antioxidants, osmolality adjusting agents, stability agents, bulking agents, emulsifying agents, wetting agents, sweetening or flavoring agents, physiological substances, dermal fillers, pharmacological substances, and the like. Various buffers and means for adjusting pH can be used to prepare a pharmaceutical composition disclosed herein, provided that the resulting preparation is pharmaceutically acceptable. Non-limiting examples of buffers include acetate buffers, borate buffers, citrate buffers, neutral buffered salines, phosphate buffers, and phosphate buffered saline. Any concentration of a buffer can be used, such as about 0.1 mM to about 900 mM. In some embodiments, an HA composition may have a pH of about 5.0 to about 8.5, about 5.0 to about 8.0, about 6.5 to about 7.5, about 7.0 to about 7.4, or about 7.1 to about 7.3. It is understood that acids or bases can be used to adjust the pH of a composition as needed.

Useful preservatives include, without limitation, benzalkonium chloride, chlorobutanol, thimerosal, phenylmercuric acetate, phenylmercuric nitrate, a stabilized oxy chloro composition, such as, e.g., sodium chlorite and chelants, such as, e.g., DTPA or DTPA-bisamide, calcium DTPA, and CaNaDTPA-bisamide. Tonicity adjustors useful in a composition disclosed herein include, without limitation, salts such as, e.g., sodium chloride or potassium chloride, mannitol, or glycerin and other pharmaceutically acceptable tonicity adjustor. A salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. Useful antioxidants include, without limitation, sodium metabisulfite, sodium thiosulfate, acetylcysteine, butylated hydroxyanisole, and butylated hydroxytoluene.

Useful anti-oxidants include, without limitation, vitamin C (ascorbic acid) and Vitagen (3-aminopropyl-L-ascorbylphosphate), a protected form of vitamin C. Any suitable amount of anti-oxidant may be used, such as about 0.01% w/w to about 2% w/w, about 0.1% w/w to about 1% w/w, about 0.5% w/w to about 0.7% w/w, or about 0.6% w/w.

Osmolality agents may be used to adjust osmolality. Useful osmolality agents include, without limitation, sodium chloride and potassium chloride; and glycerin. A composition disclosed herein exhibits a physiologically-acceptable osmolality. Osmolality refers to the concentration of osmotically active solutes per kilo of solvent in the body. A physiologically-acceptable osmolality refers to an osmolality in accord with, or characteristic of, the normal functioning of a living organism. As such, administration of a composition disclosed herein exhibits an osmolality that has substantially no long term or permanent detrimental effect when administered to a mammal. Osmolality is expressed in terms of osmoles of osmotically active solute per kilogram of solvent (osmol/kg or Osm/kg) and is equal to the sum of the molalities of all the solutes present in that solution. The osmolality of a solution can be measured using an osmometer. The most commonly used instrument in modern laboratories is a freezing point depression osmometer. This instrument measure the change in freezing point that occurs in a solution with increasing osmolality (freezing point depression osmometer) or the change in vapor pressure that occurs in a solution with increasing osmolality (vapor pressure depression osmometer).

In an embodiment, a composition disclosed herein exhibits a physiologically-acceptable osmolality. In aspects of this embodiment, a composition disclosed herein exhibits an osmolality of, e.g., about 100 mOsm/kg, about 150 mOsm/kg, about 200 mOsm/kg, about 250 mOsm/kg, about 300 mOsm/kg, about 350 mOsm/kg, about 400 mOsm/kg, about 450 mOsm/kg, or about 500 mOsm/kg. In other aspects of this embodiment, a composition disclosed herein exhibits an osmolality of, e.g., at least 100 mOsm/kg, at least 150 mOsm/kg, at least 200 mOsm/kg, at least 250 mOsm/kg, at least 300 mOsm/kg, at least 350 mOsm/kg, at least 400 mOsm/kg, at least 450 mOsm/kg, or at least 500 mOsm/kg. In yet other aspects of this embodiment, a composition disclosed herein exhibits an osmolality of, e.g., at most 100 mOsm/kg, at most 150 mOsm/kg, at most 200 mOsm/kg, at most 250 mOsm/kg, at most 300 mOsm/kg, at most 350 mOsm/kg, at most 400 mOsm/kg, at most 450 mOsm/kg, or at most 500 mOsm/kg. In still other aspects of this embodiment, a composition disclosed herein exhibits an osmolality of, e.g., about 100 mOsm/kg to about 500 mOsm/kg, about 200 mOsm/kg to about 500 mOsm/kg, about 200 mOsm/kg to about 400 mOsm/kg, about 300 mOsm/kg to about 400 mOsm/kg, about 270 mOsm/kg to about 390 mOsm/kg, about 225 mOsm/kg to about 350 mOsm/kg, about 250 mOsm/kg to about 325 mOsm/kg, about 275 mOsm/kg to about 300 mOsm/kg, or about 285 mOsm/kg to about 290 mOsm/kg.

A stability agent useful to stabilize a composition disclosed herein includes pyruvate and its salts such as sodium salts, potassium salts, lithium salts, magnesium salts, calcium salts, etc. For example, pyruvate may stabilize HA polymers and/or other components in the composition. Any suitable amount of pyruvate may be used, such as about 0.05% w/w to about 2% w/w; about 0.05% w/w to about 1% w/w; about 0.1% w/w to about 1% w/w; about 0.01% w/w to about 2% w/w; about 0.01% w/w, about 0.05% w/w, about 0.1% w/w, about 0.25% w/w, about 0.5% w/w, about 1% w/w, or any amount in a range bounded by, or between, any of these values.

A composition disclosed herein may optionally comprise one or more dermal filler materials useful as a filler for tissue space. Exemplary filler materials suitable with the compositions and methods disclosed herein include, without limitation, fillers comprising a polypeptide such as, e.g., a silk protein, (like silk fibroin), a resilin, a resilin-like polypeptide, an elastin, an elastin-like polypeptide, a silk protein-elastin-like polypeptide, an abductin, a byssus, a gliadin, a glutenin, abductin, keratin, gelatin, or collagen; fillers comprising a polysaccharide such as, e.g., cellulose, agarose, chitosan, or chitin; and fillers comprising a polyester such as, e.g., D-lactic acid, L-lactic acid, racemic lactic acid, glycolic acid, caprolactone. These additional tissue space fillers may be administered as a separate component, or may be complexed with HA polymers disclosed herein.

A composition disclosed herein may further and optionally comprise another pharmacological substance or combination of pharmacological substances that provide a beneficial effect when the composition is administered to an individual. Such beneficial agents include, without limitation, an anesthetic agent, an anti-acne agent, an anti-cellulite agent, an anti-hemorrhagic agent, an anti-inflammatory agent, an anti-irritant agent, an anti-itch agent, an anti-microbial agent, an anti-pigmentation agent, an anti-scarring agent, a desquamating agent, an immunosuppressive agent, a moisturizing agent, a pigmentation agent, a tensioning agent, a vasoconstrictor, a vasodilator, like a hemostatic agent or anti-fibrinolytic agent. Such pharmacological substances are known in the art and described in, e.g., U.S. Pat. Nos. 8,946,192, 9,114,188, 9,333,160, and 9,655,991 and US Publication 2011/0171310, the content of each of which is hereby incorporated by reference.

In aspects of this embodiment, a composition disclosed herein comprises an anesthetic agent, an anti-acne agent, an anti-cellulite agent, an anti-hemorrhagic agent, an anti-inflammatory agent, an anti-irritant agent, an anti-itch agent, an anti-microbial agent, an anti-pigmentation agent, an anti-scarring agent, a desquamating agent, an immunosuppressive agent, a moisturizing agent, a pigmentation agent, a tensioning agent, a vasoconstrictor, and/or a vasodilator in an amount of, e.g., about 0.1%, about 0.2%, about 0.3%, about 0.4%, about 0.5%, about 0.6%, about 0.7%, about 0.8% about 0.9%, about 1.0%, about 2.0%, about 3.0%, about 4.0%, about 5.0%, about 6.0%, about 7.0%, about 8.0%, about 9.0%, or about 10% by weight of the total composition. In other aspects, a composition disclosed herein comprises an anesthetic agent, an anti-acne agent, an anti-cellulite agent, an anti-hemorrhagic agent, an anti-inflammatory agent, an anti-irritant agent, an anti-itch agent, an anti-microbial agent, an anti-pigmentation agent, an anti-scarring agent, a desquamating agent, an immunosuppressive agent, a moisturizing agent, a pigmentation agent, a tensioning agent, a vasoconstrictor, and/or a vasodilator in an amount of, e.g., at least 0.1%, at least 0.2%, at least 0.3%, at least 0.4%, at least 0.5%, at least 0.6%, at least 0.7%, at least 0.8% at least 0.9%, at least 1.0%, at least 2.0%, at least 3.0%, at least 4.0%, at least 5.0%, at least 6.0%, at least 7.0%, at least 8.0%, at least 9.0%, or at least 10% by weight of the total composition. In yet other aspects, a composition disclosed herein comprises an anesthetic agent, an anti-acne agent, an anti-cellulite agent, an anti-hemorrhagic agent, an anti-inflammatory agent, an anti-irritant agent, an anti-itch agent, an anti-microbial agent, an anti-pigmentation agent, an anti-scarring agent, a desquamating agent, an immunosuppressive agent, a moisturizing agent, a pigmentation agent, a tensioning agent, a vasoconstrictor, and/or a vasodilator in an amount of, e.g., at most 0.1%, at most 0.2%, at most 0.3%, at most 0.4%, at most 0.5%, at most 0.6%, at most 0.7%, at most 0.8% at most 0.9%, at most 1.0%, at most 2.0%, at most 3.0%, at most 4.0%, at most 5.0%, at most 6.0%, at most 7.0%, at most 8.0%, at most 9.0%, or at most 10% by weight of the total composition. In further aspects, a composition disclosed herein comprises an anesthetic agent, an anti-acne agent, an anti-cellulite agent, an anti-hemorrhagic agent, an anti-inflammatory agent, an anti-irritant agent, an anti-itch agent, an anti-microbial agent, an anti-pigmentation agent, an anti-scarring agent, a desquamating agent, an immunosuppressive agent, a moisturizing agent, a pigmentation agent, a tensioning agent, a vasoconstrictor, and/or a vasodilator in an amount of, e.g., about 0.1% to about 0.5%, about 0.1% to about 1.0%, about 0.1% to about 2.0%, about 0.1% to about 3.0%, about 0.1% to about 4.0%, about 0.1% to about 5.0%, about 0.2% to about 0.9%, about 0.2% to about 1.0%, about 0.2% to about 2.0%, about 0.5% to about 1.0%, or about 0.5% to about 2.0% by weight of the total composition.

In other aspects of this embodiment, a composition disclosed herein comprises a composition disclosed herein comprises an anesthetic agent, an anti-acne agent, an anti-cellulite agent, an anti-hemorrhagic agent, an anti-inflammatory agent, an anti-irritant agent, an anti-itch agent, an anti-microbial agent, an anti-pigmentation agent, an anti-scarring agent, a desquamating agent, an immunosuppressive agent, a moisturizing agent, a pigmentation agent, a tensioning agent, a vasoconstrictor, and/or a vasodilator in an amount of, e.g., about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, about 0.09 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 2.0 mg/mL, about 3.0 mg/mL, about 4.0 mg/mL, about 5.0 mg/mL, about 6.0 mg/mL, about 7.0 mg/mL, about 8.0 mg/mL, about 9.0 mg/mL, or about 10 mg/mL. In yet other aspects of this embodiment, a composition disclosed herein comprises an anesthetic agent, an anti-acne agent, an anti-cellulite agent, an anti-hemorrhagic agent, an anti-inflammatory agent, an anti-irritant agent, an anti-itch agent, an anti-microbial agent, an anti-pigmentation agent, an anti-scarring agent, a desquamating agent, an immunosuppressive agent, a moisturizing agent, a pigmentation agent, a tensioning agent, a vasoconstrictor, and/or a vasodilator in an amount of, e.g., at least 0.01 mg/mL, at least 0.02 mg/mL, at least 0.03 mg/mL, at least 0.04 mg/mL, at least 0.05 mg/mL, at least 0.06 mg/mL, at least 0.07 mg/mL, at least 0.08 mg/mL, at least 0.09 mg/mL, at least 0.1 mg/mL, at least 0.2 mg/mL, at least 0.3 mg/mL, at least 0.4 mg/mL, at least 0.5 mg/mL, at least 0.6 mg/mL, at least 0.7 mg/mL, at least 0.8 mg/mL, at least 0.9 mg/mL, at least 1.0 mg/mL, at least 2.0 mg/mL, at least 3.0 mg/mL, at least 4.0 mg/mL, at least 5.0 mg/mL, at least 6.0 mg/mL, at least 7.0 mg/mL, at least 8.0 mg/mL, at least 9.0 mg/mL, or at least 10 mg/mL. In still other aspects of this embodiment, a composition disclosed herein comprises an anesthetic agent, an anti-acne agent, an anti-cellulite agent, an anti-hemorrhagic agent, an anti-inflammatory agent, an anti-irritant agent, an anti-itch agent, an anti-microbial agent, an anti-pigmentation agent, an anti-scarring agent, a desquamating agent, an immunosuppressive agent, a moisturizing agent, a pigmentation agent, a tensioning agent, a vasoconstrictor, and/or a vasodilator in an amount of, e.g., at most 0.01 mg/mL, at most 0.02 mg/mL, at most 0.03 mg/mL, at most 0.04 mg/mL, at most 0.05 mg/mL, at most 0.06 mg/mL, at most 0.07 mg/mL, at most 0.08 mg/mL, at most 0.09 mg/mL, at most 0.1 mg/mL, at most 0.2 mg/mL, at most 0.3 mg/mL, at most 0.4 mg/mL, at most 0.5 mg/mL, at most 0.6 mg/mL, at most 0.7 mg/mL, at most 0.8 mg/mL, at most 0.9 mg/mL, at most 1.0 mg/mL, at most 2.0 mg/mL, at most 3.0 mg/mL, at most 4.0 mg/mL, at most 5.0 mg/mL, at most 6.0 mg/mL, at most 7.0 mg/mL, at most 8.0 mg/mL, at most 9.0 mg/mL, or at most 10 mg/mL. In further aspects, a composition disclosed herein comprises an anesthetic agent, an anti-acne agent, an anti-cellulite agent, an anti-hemorrhagic agent, an anti-inflammatory agent, an anti-irritant agent, an anti-itch agent, an anti-microbial agent, an anti-pigmentation agent, an anti-scarring agent, a desquamating agent, an immunosuppressive agent, a moisturizing agent, a pigmentation agent, a tensioning agent, a vasoconstrictor, and/or a vasodilator at a concentration of, e.g., about 0.01 mg/mL to about 0.7 mg/mL, about 0.06 mg/mL to about 0.7 mg/mL, about 0.01 mg/mL to about 1.0 mg/mL, about 0.05 mg/mL to about 1.0 mg/mL, about 0.06 mg/mL to about 1.0 mg/mL, about 0.1 mg/mL to about 1.0 mg/mL, about 0.1 mg/mL to about 2.0 mg/mL, about 0.1 mg/mL to about 3.0 mg/mL, about 0.1 mg/mL to about 4.0 mg/mL, about 0.1 mg/mL to about 5.0 mg/mL, about 0.2 mg/mL to about 0.9 mg/mL, about 0.2 mg/mL to about 1.0 mg/mL, about 0.2 mg/mL to about 2.0 mg/mL, about 0.5 mg/mL to about 1.0 mg/mL, or about 0.5 mg/mL to about 2.0 mg/mL.

An anesthetic agent may be a local anesthetic agent, including an anesthetic agent that causes a reversible local anesthesia or a loss of nociception, such as, e.g., aminoamide local anesthetics and aminoester local anesthetics. Non-limiting examples of anesthetic agents may include lidocaine, ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cyclomethycaine, dibucaine, dimethisoquin, dimethocaine, diperodon, dicyclomine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, combinations thereof, and salts thereof. Non-limiting examples of aminoester local anesthetics include procaine, chloroprocaine, cocaine, cyclomethycaine, dimethocaine (larocaine), propoxycaine, procaine (novocaine), proparacaine, tetracaine (amethocaine). Non-limiting examples of aminoamide local anesthetics include articaine, bupivacaine, cinchocaine (dibucaine), etidocaine, levobupivacaine, lidocaine (lignocaine), mepivacaine, piperocaine, prilocaine, ropivacaine, trimecaine, or a combination thereof.

The amount of an anesthetic agent included may be an amount effective to reduce pain experienced by an individual upon administration of a composition disclosed herein, such as about 0.1%, about 0.2%, about 0.3%, about 0.4%, about 0.5%, about 0.6%, about 0.7%, about 0.8% about 0.9%, about 1.0%, about 2.0%, about 3.0%, about 4.0%, about 5.0%, about 6.0%, about 7.0%, about 8.0%, about 9.0%, about 10%, at least about 0.1%, at least about 0.2%, at least about 0.3%, at least about 0.4%, at least about 0.5%, at least about 0.6%, at least about 0.7%, at least about 0.8% at least about 0.9%, at least about 1.0%, at least about 2.0%, at least about 3.0%, at least about 4.0%, at least about 5.0%, at least about 6.0%, at least about 7.0%, at least about 8.0%, at least about 9.0%, at least about 10%, at most about 0.1%, at most about 0.2%, at most about 0.3%, at most about 0.4%, at most about 0.5%, at most about 0.6%, at most about 0.7%, at most about 0.8% at most about 0.9%, at most about 1.0%, at most about 2.0%, at most about 3.0%, at most about 4.0%, at most about 5.0%, at most about 6.0%, at most about 7.0%, at most about 8.0%, at most about 9.0%, at most about 10%, about 0.1% to about 0.5%, about 0.1% to about 1.0%, about 0.1% to about 2.0%, about 0.1% to about 3.0%, about 0.1% to about 4.0%, about 0.1% to about 5.0%, about 0.2% to about 0.9%, about 0.2% to about 1.0%, about 0.2% to about 2.0%, about 0.5% to about 1.0%, or about 0.5% to about 2.0%. In some embodiments, a composition disclosed herein may comprise lidocaine, in free base or salt form (e.g. lidocaine HCl) in an amount of about 0.05% w/w to about 1% w/w; about 0.1% w/w to about 0.5% w/w, or about 0.3% w/w.

A composition disclosed herein is suitable for use as a dermal filler. In some embodiments, a composition disclosed herein is a hydrogel having an appropriate elastic modulus and viscous modulus, so as to be suitable as a dermal filler.

The elastic modulus of an object includes the slope of its stress-strain curve in the elastic deformation region: λ=stress/strain, where λ is the elastic modulus in Pascal's (Pa); stress is the force causing the deformation divided by the area to which the force is applied; and strain is the ratio of the change caused by the stress to the original state of the object. Although depending on the speed at which the force is applied, a stiffer composition will have a higher elastic modulus and it will take a greater force to deform the material a given distance, such as, e.g., an injection. Specifying how stresses are to be measured, including directions, may allow many types of elastic moduli to be defined.

Viscous modulus is also known as the loss modulus because it describes the energy that is lost as viscous dissipation. Tan δ is the ratio of the viscous modulus and the elastic modulus, Tan δ=G″IG′. For Tan δ values disclosed in the present specification, a Tan δ is obtained from the dynamic modulus at a frequency of 1 Hz. A lower tan δ corresponds to a stiffer, harder, or more elastic composition.

A composition disclosed herein can be formulated into any form that enables topical application of a composition disclosed herein in a manner that achieves a desired beneficial effect. In some embodiments, a composition disclosed herein can be formulated into, e.g., a single-phase formulation, a biphasic formulation comprising a medium phase and a dispersed phase, or a multiphasic formulation. In another embodiment, a composition disclosed herein can be formulated into, e.g., a liquid composition, a colloidal composition, a semi-solid composition, or a solid composition. In another embodiment, a composition disclosed herein can be formulated into, e.g., a liquid aerosol, a foam, an emulsion, a gel, a sol, or a solid sol. In another embodiment, a composition disclosed herein can be formulated into, e.g., a spray, a liquid aerosol, a wash, an aftershave, a perfume, a body mist, a lotion, a cream, a salve, a waxing composition, a mousse, a shampoo, a conditioner, an ointment, a soap, a toner, a foundation, a stick, a film forming product, a suspension, or an emollient. In some embodiments, enables topical application of a composition disclosed herein can optionally be done in conjunction with ultrasound.

A composition disclosed herein can be formulated into any form that enables injectable application of a composition disclosed herein in a manner that achieves a desired beneficial effect. In some embodiments, a composition disclosed herein can be formulated to be administered into a skin region of an individual by injection through a fine needle, such as a needle that is about 27 gauge or smaller. In some embodiments, an HA composition is injectable through a needle of, e.g., about 27 gauge; about 30 gauge; about 32 gauge; about 22 gauge or smaller; about 27 gauge or smaller; about 30 gauge or smaller; about 32 gauge or smaller; about 22 gauge to about 35 gauge; about 22 gauge to about 34 gauge; about 22 gauge to about 33 gauge; about 22 gauge to about 32 gauge; about 22 gauge to about 27 gauge; or about 27 gauge to about 32 gauge.

A composition disclosed herein is typically resistant to biodegradation upon administration to an individual. As used herein, the term “resistant to biodegradation” is synonymous with “resistant to bioerosion”, “resistant to bioresorption”, “non-biodegradable”, “non-bioerodable” and “non-bioresorbable” and refers to a composition disclosed herein that is not prone to degrading, eroding, resorbing, decomposing, or breaking down to any substantial or significant degree while implanted in an individual. Non-limiting examples of substantial non-degradation or resistance to biodegradation include less than 10% degradation of a composition over a time period measured, less than 5% degradation of a composition over a time period measured, less than 3% degradation of a composition over a time period measured, less than 1% degradation of a composition over a time period measured. In an embodiment, a composition disclosed herein is substantially non-biodegradable or resistant to biodegradation upon administration to an individual.

In aspects of this embodiment, a composition disclosed herein is substantially non-biodegradable or resistance to biodegradation for, e.g., about 10 days, about 20 days, about 30 days, about 40 days, about 50 days, about 60 days, about 70 days, about 80 days, or about 90 days, before biodegradation occurs. In other aspects of this embodiment, a composition disclosed herein is substantially non-biodegradable or resistant to biodegradation for, e.g., at least 10 days, at least 20 days, at least 30 days, at least 40 days, at least 50 days, at least 60 days, at least 70 days, at least 80 days, or at least 90 days, before biodegradation occurs. In yet other aspects of this embodiment, a composition disclosed herein is substantially non-biodegradable or resistant to biodegradation for, e.g., at most 10 days, at most 20 days, at most 30 days, at most 40 days, at most 50 days, at most 60 days, at most 70 days, at most 80 days, or at most 90 days, before biodegradation occurs. In still other aspects of this embodiment, a composition disclosed herein is substantially non-biodegradable or resistant to biodegradation for, e.g., about 10 days to about 30 days, about 20 days to about 50 days, about 40 days to about 60 days, about 50 days to about 80 days, or about 60 days to about 90 days, before biodegradation occurs.

Aspects of the present specification provide, in part, a method of treating a soft tissue condition of an individual by administering a composition disclosed herein. Other aspects of the present specification provide, in part, a composition disclosed herein for use in treating a soft tissue condition of an individual. Other aspects of the present specification provide, in part, use of a composition disclosed herein for treating a soft tissue condition of an individual. Other aspects of the present specification provide, in part, use of a composition disclosed herein in the manufacture of a medicament for the treatment a soft tissue condition of an individual.

As used herein, the term “treating,” refers to reducing or eliminating in an individual a cosmetic or clinical symptom of a soft tissue condition characterized by a soft tissue imperfection, defect, disease, and/or disorder; or delaying or preventing in an individual the onset of a cosmetic or clinical symptom of a condition characterized by a soft tissue imperfection, defect, disease, and/or disorder; or improving and/or supporting the appearance, beauty and/or health of a skin region by restoring, maintaining and/or improving of any attribute or characteristic of a skin region, including improvement of one or more symptoms of dermatological aging and/or environmental stress. For example, the term “treating” can mean reducing a symptom of a condition characterized by a soft tissue defect, disease, and/or disorder by, e.g., at least 20%, at least 30%, at least 40%, at least 50%, at least 60%, at least 70%, at least 80%, at least 90% or at least 100%. The effectiveness of a compound disclosed herein in treating a condition characterized by a soft tissue defect, disease, and/or disorder can be determined by observing one or more cosmetic, clinical symptoms, and/or physiological indicators associated with the condition. An improvement in a soft tissue defect, disease, and/or disorder also can be indicated by a reduced need for a concurrent therapy. Those of skill in the art will know the appropriate symptoms or indicators associated with specific soft tissue defect, disease, and/or disorder and will know how to determine if an individual is a candidate for treatment with a compound or composition disclosed herein.

The composition and methods and uses disclosed herein are useful in treating a soft tissue condition. A soft tissue condition includes, without limitation, a soft tissue imperfection, defect, disease, and/or disorder. Non-limiting examples of a soft tissue condition include breast imperfection, defect, disease and/or disorder, such as, e.g., a breast augmentation, a breast reconstruction mastopexy, micromastia, thoracic hypoplasia, Poland's syndrome, defects due to implant complications like capsular contraction and/or rupture; a facial imperfection, defect, disease or disorder, such as, e.g., a facial augmentation, a facial reconstruction, a mesotherapy, Parry-Romberg syndrome, lupus erythematosus profundus, dermal divots, sunken cheeks, thin lips, nasal imperfections or defects, retro-orbital imperfections or defects, a facial fold, line and/or wrinkle like a glabellar line, a nasolabial line, a perioral line, and/or a marionette line, and/or other contour deformities or imperfections of the face; a neck imperfection, defect, disease or disorder; a skin imperfection, defect, disease and/or disorder; other soft tissue imperfections, defects, diseases and/or disorders, such as, e.g., an augmentation or a reconstruction of the upper arm, lower arm, hand, shoulder, back, torso including abdomen, buttocks, upper leg, lower leg including calves, foot including plantar fat pad, eye, genitals, or other body part, region or area, or a disease or disorder affecting these body parts, regions or areas. As used herein, the term “mesotherapy” includes a non-surgical cosmetic treatment technique of the skin involving intra-epidermal, intra-dermal, and/or subcutaneous injection of an agent administered as small multiple droplets into the epidermis, dermo-epidermal junction, and/or the dermis.

Aspects of the present specification provide, in part, a method of treating cellular senescence in a skin region of an individual by administering a composition disclosed herein. Other aspects of the present specification provide, in part, a composition disclosed herein for use in treating cellular senescence in a skin region of an individual. Other aspects of the present specification provide, in part, use of a composition disclosed herein for treating cellular senescence in a skin region of an individual. Other aspects of the present specification provide, in part, use of a composition disclosed herein in the manufacture of a medicament for the treatment cellular senescence in a skin region of an individual.

As used herein, the term “treating,” refers to reducing, eliminating, or reversing in an individual a cosmetic or clinical symptom of cellular senescence; or delaying or preventing in an individual the onset of a cosmetic or clinical symptom of cellular senescence. For example, the term “treating” can mean reducing a symptom of a condition characterized by cellular senescence by, e.g., at least 20%, at least 30%, at least 40%, at least 50%, at least 60%, at least 70%, at least 80%, at least 90% or at least 100%. The effectiveness of a compound disclosed herein in treating cellular senescence can be determined by observing one or more cosmetic, clinical symptoms, and/or physiological indicators associated with cellular senescence. An improvement one or more cosmetic, clinical symptoms, and/or physiological indicators associated with cellular senescence also can be indicated by a reduced need for a concurrent therapy. Those of skill in the art will know the appropriate symptoms or indicators associated with cellular senescence and will know how to determine if an individual is a candidate for treatment with a compound or composition disclosed herein.

The composition and methods and uses disclosed herein are useful in treating cellular senescence. Cellular senescence is permanent cell cycle arrest in damaged cells resulting in arrest of growth, resistance to apoptosis and altered differentiation functions. It has been shown to be produced by cellular stress, mitochondrial dysfunction, chemotherapy, radiation therapy, and oncogene activation.

A composition or compound is administered to an individual. An individual refers to any mammal including a human, a horse, a cow, a sheep, a dog and a cat. As such, a method disclosed herein is for human use as well as veterinarian use. An individual is typically a human and a human can be a patient. Typically, any individual who is a candidate for a conventional dermal filler procedure is a candidate for a method or use disclosed herein. In addition, the presently disclosed compositions and methods and uses may apply to individuals seeking a small/moderate enlargement, shape change or contour alteration of a body part or region, which may not be technically possible or aesthetically acceptable with existing soft tissue implant technology. Pre-operative evaluation typically includes routine history and physical examination in addition to thorough informed consent disclosing all relevant risks and benefits of the procedure.

The amount of a composition disclosed herein used may be determined based on the alteration and/or improvement desired, the reduction and/or elimination of a soft tissue condition symptom desired, the clinical and/or cosmetic effect desired by the individual and/or physician, and the body part or region being treated. The effectiveness of composition administration may be manifested by one or more of the following clinical and/or cosmetic measures: altered and/or improved soft tissue shape, altered and/or improved soft tissue size, altered and/or improved soft tissue contour, altered and/or improved tissue function, tissue ingrowth support and/or new collagen deposition, sustained engraftment of composition, improved patient satisfaction and/or quality of life, and decreased use of implantable foreign material.

For example, effectiveness of the compositions and methods and uses in treating a facial soft tissue condition may be manifested by one or more of the following clinical and/or cosmetic measures: increased and/or improved size, shape, and/or contour of facial feature like increased and/or improved size, shape, and/or contour of lip, cheek or eye region; altered size, shape, and/or contour of facial feature like altered size, shape, and/or contour of lip, cheek or eye region shape; disappearance, reduction, and/or prevention of a wrinkle, fold or line in the skin; disappearance, reduction, and/or prevention of stretch lines or marks; rehydration of the skin; increased and/or improved skin elasticity and/or resiliency; disappearance, reduction, and/or prevention of skin roughness; increased and/or improved skin thickness, plumpness, and/or tautness; increased and/or improved skin firmness; increased and/or improved skin smoothness, suppleness and/or softness; disappearance, reduction, and/or prevention of skin sagging and/or skin sinking; disappearance, reduction, and/or prevention of skin thinning; disappearance, reduction, and/or prevention of skin dryness and/or itchiness; increased and/or improved skin tone, shine, clarity, brightness, luster, and/or radiance; restoration of tone, shine, clarity, brightness, luster and/or radiance; increased and/or improved skin color, reduction or elimination of skin paleness; increased and/or improved skin optical properties by light diffraction or reflection; increased and/or improved procollagen, and/or collagen production; increased and/or improved maintenance and remodeling of elastin; increased and/or improved skin texture and/or promotion of retexturization; increased and/or improved skin barrier repair and/or function; decreased side effects; increased and/or improved patient satisfaction and/or quality of life; or any combination thereof.

As another example, effectiveness of the compositions and methods and uses in treating cellular senescence may be manifested by one or more of the following clinical and/or cosmetic measures: increased and/or improved autophagy, increased and/or improved mTOR inhibition, reduction, elimination, and/or prevention of secretion of pro-inflammatory factor, reduction, elimination, and/or prevention of pro-inflammatory chemokines, reduction, elimination, and/or prevention of matrix pro-inflammatory metalloproteinases, reduction, elimination, and/or prevention of pro-inflammatory growth factor, reduction and/or prevention of cellular senescence biomarkers, disappearance, reduction, elimination, and/or prevention of senescence-associated secretory phenotype biomarkers, disappearance, reduction and/or prevention of inflammaging, increased and/or improved patient satisfaction and/or quality of life, or any combination thereof.

The amount of a composition disclosed herein used with any of the methods or uses disclosed herein will typically be a therapeutically effective amount. As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. As a non-limiting example, an effective amount is an amount sufficient to increase and/or improve size, shape, and/or contour of facial feature like increase and/or improve size, shape, and/or contour of lip, cheek or eye region; alter size, shape, and/or contour of facial feature like altered size, shape, and/or contour of lip, cheek or eye region shape; reduce, eliminate, and/or prevent a wrinkle, fold or line in the skin; reduce, eliminate, and/or prevent stretch lines or marks; rehydration of the skin; increase and/or improve skin elasticity and/or resiliency; reduce, eliminate, and/or prevent skin roughness; increase and/or improve skin thickness, plumpness, and/or tautness; increase and/or improve skin firmness; increase and/or improve skin smoothness, suppleness and/or softness; reduce, eliminate, and/or prevent skin sagging and/or skin sinking; reduce, eliminate, and/or prevent skin thinning; reduce, eliminate, and/or prevent skin dryness and/or itchiness; increase and/or improve skin tone, shine, clarity, brightness, luster, and/or radiance; restoration of tone, shine, clarity, brightness, luster and/or radiance; increase and/or improve skin color, reduction, elimination, and/or prevention of skin paleness; increase and/or improve skin optical properties by light diffraction or reflection; increase and/or improve procollagen, and/or collagen production; increase and/or improve maintenance and remodeling of elastin; increase and/or improve skin texture and/or promotion of retexturization; increase and/or improve skin barrier repair and/or function; decreased side effects; increase and/or improve patient satisfaction and/or quality of life; or any combination thereof. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

As a non-limiting example, an effective amount is an amount sufficient to increase and/or improve autophagy, increase and/or improve mTOR inhibition, reduce, eliminate, and/or prevent secretion of pro-inflammatory factor, reduce, eliminate, and/or prevent pro-inflammatory chemokines, reduce, eliminate, and/or prevent matrix pro-inflammatory metalloproteinases, reduce, eliminate, and/or prevent pro-inflammatory growth factor, reduce, eliminate, and/or prevent cellular senescence biomarkers, reduce, eliminate, and/or prevent senescence-associated secretory phenotype biomarkers, reduce, eliminate, and/or prevent inflammaging, increase and/or improve patient satisfaction and/or quality of life, or any combination thereof.

In aspects of these embodiments, a composition disclosed herein is administered in an amount of, e.g., about 0.01 g, about 0.05 g, about 0.1 g, about 0.5 g, about 1 g, about 5 g, about 10 g, about 20 g, about 30 g, about 40 g, about 50 g, about 60 g, about 70 g, about 80 g, about 90 g, about 100 g, about 150 g, or about 200 g. In other aspects of these embodiments, a composition disclosed herein is administered in an amount of, e.g., at least 0.01 g, at least 0.05 g, at least 0.1 g, at least 0.5 g, at least 1 g, at least 5 g, at least 10 g, at least 20 g, at least 30 g, at least 40 g, at least 50 g, at least 60 g, at least 70 g, at least 80 g, at least 90 g, at least 100 g, at least 150 g, or at least 200 g. In yet other aspects of these embodiments, a composition disclosed herein is administered in an amount of, e.g., at most 0.01 g, at most 0.05 g, at most 0.1 g, at most 0.5 g, at most 1 g, at most 5 g, at most 10 g, at most 20 g, at most 30 g, at most 40 g, at most 50 g, at most 60 g, at most 70 g, at most 80 g, at most 90 g, at most 100 g, at most 150 g, or at most 200 g. In yet other aspects of these embodiments, a composition disclosed herein is administered in an amount of, e.g., about 0.01 g to about 0.1 g, about 0.1 g to about 1 g, about 1 g to about 10 g, about 1 g to about 20 g, about 1 g to about 30 g, about 1 g to about 40 g, about 1 g to about 50 g, about 10 g to about 50 g, about 10 g to about 100 g, about 10 g to about 200 g, about 50 g to about 200 g, about 100 g to about 200 g, about 150 g to about 200 g,

In aspects of these embodiments, a composition disclosed herein is administered in an amount of, e.g., about 0.01 mL, about 0.05 mL, about 0.1 mL, about 0.5 mL, about 1 mL, about 5 mL, about 10 mL, about 20 mL, about 30 mL, about 40 mL, about 50 mL, about 60 mL, about 70 mL, about 80 mL, about 90 mL, about 100 mL, about 150 mL, or about 200 mL. In other aspects of these embodiments, a composition disclosed herein is administered in an amount of, e.g., at least 0.01 mL, at least 0.05 mL, at least 0.1 mL, at least 0.5 mL, at least 1 mL, at least 5 mL, at least 10 mL, at least 20 mL, at least 30 mL, at least 40 mL, at least 50 mL, at least 60 mL, at least 70 mL, at least 80 mL, at least 90 mL, at least 100 mL, at least 150 mL, or at least 200 mL. In yet other aspects of these embodiments, a composition disclosed herein is administered in an amount of, e.g., at most 0.01 mL, at most 0.05 mL, at most 0.1 mL, at most 0.5 mL, at most 1 mL, at most 5 mL, at most 10 mL, at most 20 mL, at most 30 mL, at most 40 mL, at most 50 mL, at most 60 mL, at most 70 mL, at most 80 mL, at most 90 mL, at most 100 mL, at most 150 mL, or at most 200 mL. In yet other aspects of these embodiments, a composition disclosed herein is administered in an amount of, e.g., about 0.01 mL to about 0.1 mL, about 0.1 mL to about 1 mL, about 1 mL to about 10 mL, about 1 mL to about 20 mL, about 1 mL to about 30 mL, about 1 mL to about 40 mL, about 1 mL to about 50 mL, about 10 mL to about 50 mL, about 10 mL to about 100 mL, about 10 mL to about 200 mL, about 50 mL to about 200 mL, about 100 mL to about 200 mL, about 150 mL to about 200 mL.

The route of administration of composition administered to an individual patient will typically be determined based on the cosmetic and/or clinical effect desired by the individual and/or physician and the body part or region being treated. Compositions may be administered by any means known to persons of ordinary skill in the art including, without limitation, transdermal administration, subcutaneous administration, intradermal administration, or extramuscular administration. As used herein, the term “transdermal” or “transdermally” refers to a specific type administration that delivers a composition disclosed herein by absorption through the skin herein into the hypodermis (also known as the subcutis or superficial facia), a layer of tissue directly below the dermis. As used herein, the term “intradermal” or “intradermally” refers to a specific type of an extramuscularly administration that delivers a composition disclosed herein into the dermis. As used herein, the term “subcutaneous” or “subcutaneously” refers to a specific type of an extramuscularly administration that delivers a composition disclosed herein into the hypodermis (also known as the subcutis or superficial facia), a layer of tissue directly below the dermis. As used herein, the term “extramuscular” or “extramuscularly” refers to a specific route of administration that avoids direct delivery of composition disclosed herein into muscle tissue.

In some embodiments, a composition disclosed herein may be transdermally administered to a skin region of an individual by topically administering the composition onto the surface of a skin region. Such transdermal application results in delivery of a composition disclosed herein into a dermal region or a hypodermal region, such as an epidermal-dermal junction region, a papillary region, a reticular region, or any combination thereof.

In some embodiments, a composition disclosed herein may be extramuscularly administered to a skin region of an individual by injection into a dermal region or a hypodermal region, such as an epidermal-dermal junction region, a papillary region, a reticular region, or any combination thereof. In some embodiments, a composition disclosed herein may be subcutaneously administered to a skin region of an individual by injection into a hypodermal region, such as an epidermal-dermal junction region, a papillary region, a reticular region, or any combination thereof. In some embodiments, a composition disclosed herein may be intradermal administered to a skin region of an individual by injection into a dermal region.

As used herein, the term “dermal region” refers to the region of skin comprising the epidermal-dermal junction and the dermis including the superficial dermis (papillary region) and the deep dermis (reticular region). The skin is composed of three primary layers: the epidermis, which provides waterproofing and serves as a barrier to infection; the dermis, which serves as a location for the appendages of skin; and the hypodermis (subcutaneous adipose layer). The epidermis contains no blood vessels, and is nourished by diffusion from the dermis. The main type of cells which make up the epidermis are keratinocytes, melanocytes, Langerhans cells and Merkels cells.

The dermis includes the layer of skin beneath the epidermis that includes connective tissue and cushions the body from stress and strain. The dermis is tightly connected to the Evans, Compositions, Methods and Uses for Treating Skin Aging epidermis by a basement membrane. It also harbors many mechanoreceptor/nerve endings that provide the sense of touch and heat. It contains the hair follicles, sweat glands, sebaceous glands, apocrine glands, lymphatic vessels and blood vessels. The blood vessels in the dermis provide nourishment and waste removal from its own cells as well as from the Stratum basale of the epidermis. The dermis is structurally divided into two areas: a superficial area adjacent to the epidermis, called the papillary region, and a deep thicker area known as the reticular region.

The papillary region is composed of loose areolar connective tissue. It is named for its fingerlike projections called papillae that extend toward the epidermis. The papillae provide the dermis with a “bumpy” surface that interdigitates with the epidermis, strengthening the connection between the two layers of skin. The reticular region lies deep in the papillary region and is usually much thicker. It is composed of dense irregular connective tissue, and receives its name from the dense concentration of collagenous, elastic, and reticular fibers that weave throughout it. These protein fibers give the dermis its properties of strength, extensibility, and elasticity. Also located within the reticular region are the roots of the hair, sebaceous glands, sweat glands, receptors, nails, and blood vessels. Tattoo ink is held in the dermis. Stretch marks from pregnancy are also located in the dermis.

The hypodermis lies below the dermis. Its purpose is to attach the dermal region of the skin to underlying bone and muscle as well as supplying it with blood vessels and nerves. It includes loose connective tissue and elastin. The main cell types are fibroblasts, macrophages and adipocytes (the hypodermis contains 50% of body fat). Fat serves as padding and insulation for the body.

In some embodiments, methods and uses disclosed herein further comprise cross linking HA polymers after administration to an individual. Such in-situ cross-linking step allows for a more fluid composition disclosed herein to be administered and then once in the appropriate skin region, the fluid composition can be cross-linked in situ to create a more gel-like composition which increases stability and longevity of the treatment. In some embodiments, the one or more monoterpene compounds serve as a cross-linking agent that facilitates the in-situ cross-linking of the HA polymers. In some embodiments a composition disclosed herein further includes one or more cross-linking agents disclosed herein. In some embodiments, HA polymer crosslinking may optionally further include the use of ultraviolet (UV) light and/or ultrasound to facilitate the in-situ cross-linking of the HA polymers.

A composition disclosed herein can be applied according to a method or use disclosed herein as often as needed and/or desired. Frequency of treatment may be determined based on the cosmetic and/or clinical effect desired by the individual and/or physician and the body part or region being treated. A disclosed composition can be administered once, or over a plurality of times. Ultimately, the timing used will follow quality care standards. For some treatments, administration of a composition disclosed herein can effectively treat a soft tissue condition for, e.g., about 3 months, about 4 months, about 5 months, about 6 months, about 7 months, about 8 months, about 9 months, about 10 months, about 11 months, about 12 months, about 13 months, about 14 months, about 15 months, about 18 months, or about 24 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 18 months, or at least about 24 months, about 3 months to about 6 months, about 3 months to about 12 months, about 3 months to about 15 months, about 3 months to about 18 months, about 3 months to about 21 months, about 3 months to about 24 months, about 6 months to about 12 months, about 6 months to about 15 months, about 6 months to about 18 months, about 6 months to about 21 months, about 6 months to about 24 months, about 9 months to about 12 months, about 9 months to about 15 months, about 9 months to about 18 months, about 9 months to about 21 months, about 6 months to about 24 months, about 12 months to about 15 months, about 12 months to about 18 months, about 12 months to about 21 months, about 12 months to about 24 months, about 15 months to about 18 months, about 15 months to about 21 months, about 15 months to about 24 months, about 18 months to about 21 months, about 18 months to about 24 months, or about 21 months to about 24 months.

Aspects of the present specification can also be described by the following embodiments:
1. A composition comprising one or more monoterpene compounds and HA polymers.
2. The composition of embodiment 2, wherein one or more monoterpene compounds are in an amount of about 5 mM/L, about 10 mM/L, about 15 mM/L, about 20 mM/L, about 25 mM/L, about 30 mM/L, about 35 mM/L, about 40 mM/L, about 45 mM/L, about 50 mM/L, about 55 mM/L, about 60 mM/L, about 65 mM/L, about 70 mM/L, about 75 mM/L, about 80 mM/L, about 85 mM/L, about 90 mM/L, about 95 mM/L, or about 100 mM/L.
3. The composition of embodiment 2, wherein one or more monoterpene compounds are in an amount of at least 5 mM/L, at least 10 mM/L, at least 15 mM/L, at least 20 mM/L, at least 25 mM/L, at least 30 mM/L, at least 35 mM/L, at least 40 mM/L, at least 45 mM/L, at least 50 mM/L, at least 55 mM/L, at least 60 mM/L, at least 65 mM/L, at least 70 mM/L, at least 75 mM/L, at least 80 mM/L, at least 85 mM/L, at least 90 mM/L, at least 95 mM/L, at least 100 mM/L and/or in an amount of at most 5 mM/L, at most 10 mM/L, at most 15 mM/L, at most 20 mM/L, at most 25 mM/L, at most 30 mM/L, at most 35 mM/L, at most 40 mM/L, at most 45 mM/L, at most 50 mM/L, at most 55 mM/L, at most 60 mM/L, at most 65 mM/L, at most 70 mM/L, at most 75 mM/L, at most 80 mM/L, at most 85 mM/L, at most 90 mM/L, at most 95 mM/L, or at most 100 mM/L.
4. The composition of embodiment 2, wherein one or more monoterpene compounds are in an amount of about 5 mM/L to about 10 mM/L, about 5 mM/L to about 20 mM/L, about 5 mM/L to about 30 mM/L, about 5 mM/L to about 40 mM/L, about 5 mM/L to about 50 mM/L, about 5 mM/L to about 60 mM/L, about 5 mM/L to about 70 mM/L, about 5 mM/L to about 80 mM/L, about 5 mM/L to about 90 mM/L, about 5 mM/L to about 100 mM/L, about 10 mM/L to about 20 mM/L, about 10 mM/L to about 30 mM/L, about 10 mM/L to about 40 mM/L, about 10 mM/L to about 50 mM/L, about 10 mM/L to about 60 mM/L, about 10 mM/L to about 70 mM/L, about 10 mM/L to about 80 mM/L, about 10 mM/L to about 90 mM/L, about 10 mM/L to about 100 mM/L, about 20 mM/L to about 30 mM/L, about 20 mM/L to about 40 mM/L, about 20 mM/L to about 50 mM/L, about 20 mM/L to about 60 mM/L, about 20 mM/L to about 70 mM/L, about 20 mM/L to about 80 mM/L, about 20 mM/L to about 90 mM/L, about 20 mM/L to about 100 mM/L, about 30 mM/L to about 40 mM/L, about 30 mM/L to about 50 mM/L, about 30 mM/L to about 60 mM/L, about 30 mM/L to about 70 mM/L, about 30 mM/L to about 80 mM/L, about 30 mM/L to about 90 mM/L, about 30 mM/L to about 100 mM/L, about 40 mM/L to about 50 mM/L, about 40 mM/L to about 60 mM/L, about 40 mM/L to about 70 mM/L, about 40 mM/L to about 80 mM/L, about 40 mM/L to about 90 mM/L, about 40 mM/L to about 100 mM/L, about 50 mM/L to about 60 mM/L, about 50 mM/L to about 70 mM/L, about 50 mM/L to about 80 mM/L, about 50 mM/L to about 90 mM/L, about 50 mM/L to about 100 mM/L, about 60 mM/L to about 70 mM/L, about 60 mM/L to about 80 mM/L, about 60 mM/L to about 90 mM/L, about 60 mM/L to about 100 mM/L, about 70 mM/L to about 80 mM/L, about 70 mM/L to about 90 mM/L, about 70 mM/L to about 100 mM/L, about 80 mM/L to about 90 mM/L, about 80 mM/L to about 100 mM/L, or about 90 mM/L to about 100 mM/L.
5. The composition of any one of embodiments 1-4, wherein the one or more monoterpene compounds include a monoterpene, a derivative of a monoterpene, a monoterpenoid, a derivative of a monoterpenoid, or any combination thereof.
6. The composition of embodiment 5, wherein the monoterpene is a linear monoterpene, a monocyclic monoterpene, a bicyclic monoterpene, a tricyclic monoterpene, or any combination thereof.
7. The composition of embodiment 5 or 6, wherein the derivative of a monoterpene is a carbamate, an ester, an ether, an alcohol or an aldehyde of a linear monoterpene, a carbamate, an ester, an ether, an alcohol or an aldehyde of a monocyclic monoterpene, a carbamate, an ester, an ether, an alcohol or an aldehyde of a bicyclic monoterpene, a carbamate, an ester, an ether, an alcohol or an aldehyde of a tricyclic monoterpene, or any combination thereof.
8. The composition of embodiment 6 or 7, wherein the linear monoterpene is α-myrcene, β-myrcene, α-ocimene, β-ocimene, or any combination thereof.
9. The composition of any one of embodiments 6-8, wherein the monocyclic monoterpene is limonene, α-phellandrene, β-phellandrene, α-terpineol, β-terpineol, γ-terpineol, 4-terpineol, terpinen-4-ol, α-terpinene, β-terpinene, γ-terpinene, δ-terpinene (terpinolene), or any combination thereof.
10. The composition of any one of embodiments 6-9, wherein the bicyclic monoterpene is camphene, carene, myrtersol, myrtenal, α-pinene, β-pinene, pinocarveol, sabinene, thujene, verbanol, verbanon, or any combination thereof.
11. The composition of any one of embodiments 6-10, wherein the tricyclic monoterpene include tricyclene.
12. The composition of any one of embodiments 5-11, wherein the monoterpenoid is a linear monoterpenoid, a monocyclic monoterpenoid, a bicyclic monoterpenoid, a tricyclic monoterpenoid, or any combination thereof.
13. The composition of any one of embodiments 5-12, wherein the derivative of a monoterpenoid is a carbamate, an ester, an ether, an alcohol or an aldehyde of a linear monoterpenoid, a carbamate, an ester, an ether, an alcohol or an aldehyde of a monocyclic monoterpenoid, a carbamate, an ester, an ether, an alcohol or an aldehyde of a bicyclic monoterpenoid, a carbamate, an ester, an ether, an alcohol or an aldehyde of a tricyclic monoterpenoid, or any combination thereof.
14. The composition of embodiment 12 or 13, wherein the linear monoterpenoid is citral, citronellal, citronellol, geranial (citral A), geraniol, geranyl pyrophosphate, halomon, linalool, myrcenol, nerol (citral B), or any combination thereof.
15. The composition of any one of embodiments 12-14, wherein the monocyclic monoterpenoid is carvacrol, p-cymene, grapefruit mercaptan, menthol, perillyl alcohol, α-thujaplicin, β-thujaplicin (hinokitiol), γ-thujaplicin, thymol, or any combination thereof.
16. The composition of any one of embodiments 12-15, wherein the bicyclic monoterpenoid is ascaridole, borneol, bornyl acetate, camphor, eucalyptol, umbellulone, or any combination thereof.
17. The composition of any one of embodiments 1-16, wherein the HA polymers are in an amount of about 1%, about 2%, about 3%, about 4%, about 5%, about 6%, about 7%, about 8%, or about 9%, or about 10% by weight of the composition.
18. The composition of any one of embodiments 1-16, wherein the HA polymers are in an amount of at least 1%, at least 2%, at least 3%, at least 4%, at least 5%, at least 6%, at least 7%, at least 8%, at least 9%, or at least 10% by weight of the composition and/or at most 1%, at most 2%, at most 3%, at most 4%, at most 5%, at most 6%, at most 7%, at most 8%, at most 9%, or at most 10% by weight of the composition.
19. The composition of any one of embodiments 1-16, wherein the HA polymers are in an amount of about 1% to about 10%, about 2% to about 10%, about 3% to about 10%, about 4% to about 10%, about 5% to about 10%, about 6% to about 10%, about 7% to about 10%, about 1% to about 9%, about 2% to about 9%, about 3% to about 9%, about 4% to about 9%, about 5% to about 9%, about 6% to about 9%, or about 7% to about 9%, about 1% to about 8%, about 2% to about 8%, about 3% to about 8%, about 4% to about 8%, about 5% to about 8%, about 6% to about 8%, about 1% to about 7%, about 2% to about 7%, about 3% to about 7%, about 4% to about 7%, about 5% to about 7%, about 1% to about 6%, about 2% to about 6%, about 3% to about 6%, about 4% to about 6%, about 1% to about 5%, about 2% to about 5%, about 3% to about 5%, about 1% to about 4%, about 2% to about 4%, about 1% to about 3%, by weight of the composition.
20. The composition of any one of embodiments 1-19, wherein the HA polymers are in an amount of about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 13.5 mg/mL, about 14 mg/mL, about 15 mg/mL, about 16 mg/mL, about 17 mg/mL, about 18 mg/mL, about 19 mg/mL, or about 20 mg/mL.
21. The composition of any one of embodiments 1-19, wherein the HA polymers are in an amount of at least 1 mg/mL, at least 2 mg/mL, at least 3 mg/mL, at least 4 mg/mL, at least 5 mg/mL, at least 10 mg/mL, at least 15 mg/mL, at least 20 mg/mL, at least 25 mg/mL, at least 30 mg/mL, at least 35 mg/mL, or at least 40 mg/mL and/or in an amount of at most 1 mg/mL, at most 2 mg/mL, at most 3 mg/mL, at most 4 mg/mL, at most 5 mg/mL, at most 10 mg/mL, at most 15 mg/mL, at most 20 mg/mL, at most 25 mg/mL, at most 30 mg/mL, at most 35 mg/mL, or at most 40 mg/mL
22. The composition of any one of embodiments 1-19, wherein the HA polymers are in an amount of about 7.5 mg/mL to about 19.5 mg/mL, about 8.5 mg/mL to about 18.5 mg/mL, about 9.5 mg/mL to about 17.5 mg/mL, about 10.5 mg/mL to about 16.5 mg/mL, about 11.5 mg/mL to about 15.5 mg/mL, about 12.5 mg/mL to about 14.5 mg/mL, about 15 mg/mL to about 20 mg/mL, about 15 mg/mL to about 25 mg/mL, about 15 mg/mL to about 30 mg/mL, about 15 mg/mL to about 35 mg/mL, about 15 mg/mL to about 40 mg/mL, about 20 mg/mL to about 25 mg/mL, about 20 mg/mL to about 30 mg/mL, about 20 mg/mL to about 35 mg/mL, about 20 mg/mL to about 40 mg/mL, about 25 mg/mL to about 30 mg/mL, about 25 mg/mL to about 35 mg/mL, about 25 mg/mL to about 40 mg/mL, about 30 mg/mL to about 35 mg/mL, about 30 mg/mL to about 40 mg/mL, or about 35 mg/mL to about 40 mg/mL
23. The composition of any one of embodiments 1-22, wherein the HA polymers comprise low molecular weight HA polymers, high molecular weight HA polymers, or both low molecular weight HA polymers and high molecular weight HA polymers.
24. The composition of a embodiment 23, wherein the low molecular weight HA polymers have a mean molecular weight of at most 100,000 Da, at most 200,000 Da, at most 300,000 Da, at most 400,000 Da, at most 500,000 Da, at most 600,000 Da, at most 700,000 Da, at most 800,000 Da, at most 900,000 Da, or at most 950,000 Da.
25. The composition of embodiment 23 or 24, wherein the high molecular weight HA polymers have a mean molecular weight of at least 1,000,000 Da, at least 1,500,000 Da, at least 2,000,000 Da, at least 2,500,000 Da, at least 3,000,000 Da, at least 3,500,000 Da, at least 4,000,000 Da, at least 4,500,000 Da, or at least 5,000,000 Da, at least 6,000,000 Da, at least 7,000,000 Da, at least 8,000,000 Da, at least 9,000,000 Da, or at least 10,000,000 Da.
26. The composition of any one of embodiments 23-25, wherein when both low molecular weight HA polymers and high molecular weight HA polymers, the weight ratio of high molecular weight HA polymers to low molecular weight HA polymers is about 20, about 15, about 10, about 5, about 1, about 0.07, about 0.05, about 0.2, or about 0.1 to 1.
27. The composition of any one of embodiments 1-26, wherein the HA polymers comprise uncrosslinked HA polymers, crosslinked HA polymers, or both uncrosslinked HA polymers and crosslinked HA polymers.
28. The composition of embodiment 27, wherein when composition comprises a mixture of crosslinked HA polymers and uncrosslinked HA polymer the amount of crosslinked HA polymers is about 10%, about 20%, about 30%, about 40%, about 50%, about 60%, about 70%, about 80%, about 90%, about 95%, or about 100% by weight relative to the total amount of HA polymers present in the composition.
29. The composition of embodiment 27, wherein when composition comprises a mixture of crosslinked HA polymers and uncrosslinked HA polymer the amount of crosslinked HA polymers is at least 10%, at least 20%, at least 30%, at least 40%, at least 50%, at least 60%, at least 70%, at least 80%, at least 90%, at least 95%, or at least 99% by weight relative to the total amount of HA polymers present in the composition and/or at most 10%, at most 20%, at most 30%, at most 40%, at most 50%, at most 60%, at most 70%, at most 80%, at most 90%, at most 95%, or at most 99% by weight relative to the total amount of HA polymers present in the composition.
30. The composition of embodiment 27, wherein when composition comprises a mixture of crosslinked HA polymers and uncrosslinked HA polymer the amount of crosslinked HA polymers is about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 80% to about 100% by weight relative to the total amount of HA polymers present in the composition.
31. The composition of any one of embodiments 27-30, wherein the crosslinked HA polymers have a degree of crosslinking of about 1%, about 2%, about 3%, about 4%, about 5%, about 6%, about 7%, about 8%, about 9%, about 10%, about 11%, about 12%, about 13%, about 14%, or about 15%.
32. The composition of any one of embodiments 27-30, wherein the crosslinked HA polymers have a degree of crosslinking of at least 1%, at least 2%, at least 3%, at least 4%, at least 5%, at least 6%, at least 7%, at least 8%, at least 9%, at least 10%, at least 11%, at least 12%, at least 13%, at least 14%, or at least 15% and/or a degree of crosslinking of at most 1%, at most 2%, at most 3%, at most 4%, at most 5%, at most 6%, at most 7%, at most 8%, at most 9%, at most 10%, at most 11%, at most 12%, at most 13%, at most 14%, or at most 15%.
33. The composition of any one of embodiments 27-30, wherein the crosslinked HA polymers have a degree of crosslinking of about 1% to about 3%, about 1% to about 5%, about 1% to about 7%, about 1% to about 10%, about 1% to about 15%, about 3% to about 15%, about 5% to about 15%, about 7% to about 15%, about 9% to about 15%, about 10% to about 15%, about 11% to about 15%, about 2% to about 11%, about 3% to about 11%, about 4% to about 11%, about 5% to about 11%, about 6% to about 11%, about 7% to about 11%, about 8% to about 11%, about 9% to about 11%, about 3% to about 10%, about 4% to about 10%, about 5% to about 10%, about 6% to about 10%, about 7% to about 10%, about 8% to about 10%, about 4% to about 9%, about 5% to about 9%, about 6% to about 9%, about 7% to about 9%, about 5% to about 8%, or about 6% to about 8%.
34. The composition of any one of embodiments 27-33, wherein the uncrosslinked HA polymers comprise low molecular weight uncrosslinked HA polymers, high molecular weight uncrosslinked HA polymers, or both low molecular weight uncrosslinked HA polymers and high molecular weight uncrosslinked HA polymers.
35. The composition of any one of embodiments 27-34, wherein the crosslinked HA polymers comprise low molecular weight crosslinked HA polymers, high molecular weight crosslinked HA polymers, or both low molecular weight crosslinked HA polymers and high molecular weight crosslinked HA polymers.
36. The composition of any one of embodiments 27-35, wherein when both low molecular weight crosslinked HA polymers and high molecular weight HA polymers are present, low molecular weight crosslinked HA polymers are crosslinked to low molecular weight crosslinked HA polymers, high molecular weight crosslinked HA polymers are crosslinked to high molecular weight crosslinked HA polymers, low molecular weight crosslinked HA polymers are crosslinked to high molecular weight crosslinked HA polymers, or any combination thereof.
37. The composition of any one of embodiments 1-36, wherein the composition further comprises one or more monoterpene compound-HA polymer conjugates, wherein each of the one or more monoterpene compound-HA polymer conjugates comprises a monoterpene compound covalently linked to a HA polymer.
38. The composition of any one of embodiments 1-37, wherein the composition further comprises one or more carriers.
39. The composition of any one of embodiments 1-38, wherein the composition further comprises one or more other pharmaceutically acceptable components.
40. The composition of embodiment 39, wherein the one or more other pharmaceutically acceptable components comprise buffers, preservatives, tonicity adjusters, salts, antioxidants, osmolality adjusting agents, stability agents, bulking agents, emulsifying agents, wetting agents, sweetening or flavoring agents, physiological substances, dermal fillers, pharmacological substances, or any combination thereof.
41. The composition of embodiment 40, wherein the pharmacological substance comprises an anesthetic agent, an anti-acne agent, an anti-cellulite agent, an anti-hemorrhagic agent, an anti-inflammatory agent, an anti-irritant agent, an anti-itch agent, an anti-microbial agent, an anti-pigmentation agent, an anti-scarring agent, a desquamating agent, an immunosuppressive agent, a moisturizing agent, a pigmentation agent, a tensioning agent, a vasoconstrictor, a vasodilator, or any combination thereof.
42. A method of treating a soft tissue condition of an individual, the method comprising the step of administering a composition as defined in any one of embodiments 1-41.
43. A method of treating cellular senescence in a skin region of an individual, the method comprising the step of administering a composition as defined in any one of embodiments 1-41.
44. The method of embodiment 42 or 43, wherein the composition is administered in an amount of about 0.01 g, about 0.05 g, about 0.1 g, about 0.5 g, about 1 g, about 5 g, about 10 g, about 20 g, about 30 g, about 40 g, about 50 g, about 60 g, about 70 g, about 80 g, about 90 g, about 100 g, about 150 g, or about 200 g.
45. The method of embodiment 42 or 43, wherein the composition is administered in an amount of at least 0.01 g, at least 0.05 g, at least 0.1 g, at least 0.5 g, at least 1 g, at least 5 g, at least 10 g, at least 20 g, at least 30 g, at least 40 g, at least 50 g, at least 60 g, at least 70 g, at least 80 g, at least 90 g, at least 100 g, at least 150 g, or at least 200 g and/or in an amount of at most 0.01 g, at most 0.05 g, at most 0.1 g, at most 0.5 g, at most 1 g, at most 5 g, at most 10 g, at most 20 g, at most 30 g, at most 40 g, at most 50 g, at most 60 g, at most 70 g, at most 80 g, at most 90 g, at most 100 g, at most 150 g, or at most 200 g.
46. The method of embodiment 42 or 43, wherein the composition is administered in an amount of about 0.01 g to about 0.1 g, about 0.1 g to about 1 g, about 1 g to about 10 g, about 1 g to about 20 g, about 1 g to about 30 g, about 1 g to about 40 g, about 1 g to about 50 g, about 10 g to about 50 g, about 10 g to about 100 g, about 10 g to about 200 g, about 50 g to about 200 g, about 100 g to about 200 g, about 150 g to about 200 g,
47. The method of any one of embodiments 42-46, wherein the composition is administered in a volume of about 0.01 mL, about 0.05 mL, about 0.1 mL, about 0.5 mL, about 1 mL, about 5 mL, about 10 mL, about 20 mL, about 30 mL, about 40 mL, about 50 mL, about 60 mL, about 70 mL, about 80 mL, about 90 mL, about 100 mL, about 150 mL, or about 200 mL.
48. The method of any one of embodiments 42-46, wherein the composition is administered in a volume of at least 0.01 mL, at least 0.05 mL, at least 0.1 mL, at least 0.5 mL, at least 1 mL, at least 5 mL, at least 10 mL, at least 20 mL, at least 30 mL, at least 40 mL, at least 50 mL, at least 60 mL, at least 70 mL, at least 80 mL, at least 90 mL, at least 100 mL, at least 150 mL, or at least 200 mL and/or in a volume of at most 0.01 mL, at most 0.05 mL, at most 0.1 mL, at most 0.5 mL, at most 1 mL, at most 5 mL, at most 10 mL, at most 20 mL, at most 30 mL, at most 40 mL, at most 50 mL, at most 60 mL, at most 70 mL, at most 80 mL, at most 90 mL, at most 100 mL, at most 150 mL, or at most 200 mL.
49. The method of any one of embodiments 42-46, wherein the composition is administered in a volume of about 0.01 mL to about 0.1 mL, about 0.1 mL to about 1 mL, about 1 mL to about 10 mL, about 1 mL to about 20 mL, about 1 mL to about 30 mL, about 1 mL to about 40 mL, about 1 mL to about 50 mL, about 10 mL to about 50 mL, about 10 mL to about 100 mL, about 10 mL to about 200 mL, about 50 mL to about 200 mL, about 100 mL to about 200 mL, about 150 mL to about 200 mL.
50. The method of any one of embodiments 42-49, wherein the composition is administered topically onto a skin region.
51. The method of any one of embodiments 42-49, wherein the composition is administered by injection into a skin region.
52. A composition as defined in any one of embodiments 1-41 for use in treating a soft tissue condition of an individual.
53. Use of a composition as defined in any one of embodiments 1-41 for treating a soft tissue condition of an individual.
54. Use of a composition as defined in any one of embodiments 1-41 in the manufacture of a medicament for the treatment a soft tissue condition of an individual.
55. A composition as defined in any one of embodiments 1-41 for use in treating cellular senescence in a skin region of an individual.
56. Use of a composition as defined in any one of embodiments 1-41 for treating cellular senescence in a skin region of an individual.
57. Use of a composition as defined in any one of embodiments 1-41 in the manufacture of a medicament for the treatment cellular senescence in a skin region of an individual.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the compounds, pharmaceutical compositions, or methods and uses disclosed herein.

### Example 1

### Case Studies

Participant 1 was a 75-year-old Fitzpatrick grade II male with a basal cell carcinoma (BCC) of his left temple. He had previous BCC excisions and was not interested in standard treatments. Under informed consent, his physician provided him a composition comprising 0.3% perillyl alcohol and 1% HA. He topically applied this composition on a daily basis. Initially he noted redness and over the next two weeks, he began to have clinical resolution of his lesion. By 6 weeks, he no longer had a visible lesion. In addition, clinical examined by a skin expert determined that the surrounding actinic keratosis had also resolved in the region of application.

Participant 2 was a 40-year-old Fitzpatrick grade II female who noted fine lines and irregularities of pigmentation on her face. She regularly used a variety of face creams and serums with little noticeable effect. Under informed consent, her physician provided her a composition comprising 0.76% perillyl alcohol and 1% HA. She topically applied this composition on a daily basis. Initially she noted improvement in pigment irregularities and fine lines. After 6 weeks of treatment, she was clinically examined by a skin expert and it was determined that she had a 70% reduction in fine lines in the periorbital and glabellar region. In addition, her pigment irregularities were resolved. She was then transitioned to a composition comprising 0.3% perillyl alcohol and 1% HA daily application, and it was noted by a skin expert that the reduction in fine line appearance was maintained.

Participant 3 was a 31-year-old Fitzpatrick grade III female who noted early signs of aging including fine lines in the glabellar region. She only used moisturizing cream on a regular basis. Under informed consent, her physician provided her a composition comprising 0.3% perillyl alcohol and 1% HA. She topically applied this composition on a daily basis. After two weeks of treatment, she was clinically examined by a skin expert and it was determined that she showed improvement both the quality of her skin and her fine lines. This improvement was maintained when assessed a second time by the skin expert at the 6-week mark.

Participant 4 was a 29-year-old Fitzpatrick grade III female who noted fine lines and pigment irregularities. She had used simple face creams in the past without success. Under informed consent, her physician provided her a composition comprising 0.76% perillyl alcohol and 1% HA. She topically applied this composition on a daily basis. After six weeks of treatment, she was clinically examined by a skin expert and it was determined that she had a 50% reduction of fine lines. She was then transitioned to a composition comprising 0.3% perillyl alcohol and 1% HA daily application, and it was noted that she clinically maintained the reduction in fine line appearance.

Participant 5 was a 35-year-old Fitzpatrick grade II female who noted fine lines in her crow's feet region. Under informed consent, her physician provided her a composition comprising 0.3% perillyl alcohol and 1% HA. She topically applied this composition on a daily basis. After two weeks, she was clinically examined by a skin expert and it was determined that she had a 30% reduction in fine lines and by six weeks she was found to have a 50% reduction in fine lines.

Participant 6 was a 33-year-old Fitzpatrick grade II female who noted the appearance of deepening dynamic facial rhytids. Under informed consent, her physician provided her a composition comprising 0.3% perillyl alcohol and 1% HA. She topically applied this composition on a daily basis. After six weeks of treatment, she was clinically examined by a skin expert and it was determined that she had an improvement in her overall dermal thickness and a reduction in the appearance of her dynamic facial rhytids.

Participant 7 was a 75-year-old Fitzpatrick grade II female who had aging skin with deep and fine rhytids, chronic rosacea and actinic keratosis. Under informed consent, her physician provided her a composition comprising 0.3% perillyl alcohol and 1% HA. She topically applied this composition on a daily basis. After two weeks, she was clinically examined by a skin expert and it was determined that she had a 50% reduction in deep and fine rhytids. She stopped treatment because of the pronounced effect but noted a recurrence of rhytids after 6 weeks and initiated treatment again. On re-instatement of a daily topical application of a composition comprising 0.3% perillyl alcohol and 1% HA she was clinically assessed by a skin expert as having once again achieved a 50% reduction in her symptoms after two weeks of treatment.

### Example 2

### Clinical Study

This example illustrates how to optimize a formulation disclosed herein to allow for appropriate dosing and delivery of perillyl alcohol to reduce cellular senescence and improve a skin condition.

A double-blind, randomized clinical study will be conducted using human participants who are 18 years or older. The study formulations will include 1) Low Experimental Formulation, a topical formulation comprising 2% hyaluronic acid polymer and 20 mM perillyl alcohol; 2) High Experimental Formulation, a topical formulation comprising 2% hyaluronic acid polymer and 50 mM perillyl alcohol; and 3) Control Formulation, a topical formulation comprising 2% hyaluronic acid polymer without perillyl alcohol. Participants will be divided into three groups of at least five individuals. Group 1 participants will be administered the Low Experimental Formulation by applying the composition by massage on the dorsal area of each hand until completely absorbed. Group 2 participants will be administered the High Experimental Formulation by applying the composition by massage on the dorsal area of each hand until completely absorbed. Group 3 participants will be administered the Control Formulation by applying the composition by massage on the dorsal area of each hand until completely absorbed. Each group will apply their respective composition twice daily for 5 days per week.

Clinical assessment will be performed at baseline, 6-week, and 6-month time periods. This includes photographic record with standardized distance and lighting. Dermatologic assessment including skin reaction, dermal quality, reduction in pigment abnormalities, wrinkles and overall assessment of aging will be qualitatively assessed by a trained, independent study nurse. In addition, tissue analysis will be performed at same baseline, 6-week, and 6-month time periods. Analysis includes quantitative analysis cell cycle regulators p16(INK4a), p21(CIP1) and p53, cellular senescence biomarkers Lamin B1, and SA-8-galactosidase, and SASP-specific biomarkers MMP3, MM P9, IL-6, and IL-8. This will be performed by 2 mm punch biopsy. Different locations will be used at baseline, 6 weeks and 6 months to ensure that scar maturation/wound healing does not confound the results. Raman spectroscopy will be used at the same baseline, 6-week, and 6-month time periods in order to facilitate calibration of Raman spectroscopy for future studies.

The expected outcome is a significant clinical and tissue effect of perillyl alcohol. We expect a reduction in cellular senescence through mTOR inhibition by perillyl alcohol. Future studies will include a larger sample size, different amounts of perillyl alcohol, and exclusive use of Raman spectroscopy as an outcome measure. Future studies could also include a case-control comparison with rapamyacin or other related mTOR inhibitors. Eventually, based on these initial study findings, we can begin to optimize the molecular binding of POH-related molecules to reduce cellular senescence even more effectively.

In closing, foregoing descriptions of embodiments of the present invention have been presented for the purposes of illustration and description. It is to be understood that, although aspects of the present invention are highlighted by referring to specific embodiments, one skilled in the art will readily appreciate that these described embodiments are only illustrative of the principles comprising the present invention. As such, the specific embodiments are not intended to be exhaustive or to limit the invention to the precise forms disclosed. Therefore, it should be understood that embodiments of the disclosed subject matter are in no way limited to a particular element, compound, composition, component, article, apparatus, methodology, use, protocol, step, and/or limitation described herein, unless expressly stated as such.

In addition, groupings of alternative embodiments, elements, steps and/or limitations of the present invention are not to be construed as limitations. Each such grouping may be referred to and claimed individually or in any combination with other groupings disclosed herein. It is anticipated that one or more alternative embodiments, elements, steps and/or limitations of a grouping may be included in, or deleted from, the grouping for reasons of convenience and/or patentability. When any such inclusion or deletion occurs, the specification is deemed to contain the grouping as modified, thus fulfilling the written description of all Markush groups used in the appended claims.

Furthermore, those of ordinary skill in the art will recognize that certain changes, modifications, permutations, alterations, additions, subtractions and sub-combinations thereof can be made in accordance with the teachings herein without departing from the spirit of the present invention. Furthermore, it is intended that the following appended claims and claims hereafter introduced are interpreted to include all such changes, modifications, permutations, alterations, additions, subtractions and sub-combinations as are within their true spirit and scope. Accordingly, the scope of the present invention is not to be limited to that precisely as shown and described by this specification.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

The words, language, and terminology used in this specification is for the purpose of describing particular embodiments, elements, steps and/or limitations only and is not intended to limit the scope of the present invention, which is defined solely by the claims. In addition, such words, language, and terminology are to be understood not only in the sense of their commonly defined meanings, but to include by special definition in this specification structure, material or acts beyond the scope of the commonly defined meanings. Thus, if an element, step or limitation can be understood in the context of this specification as including more than one meaning, then its use in a claim must be understood as being generic to all possible meanings supported by the specification and by the word itself.

The definitions and meanings of the elements, steps or limitations recited in a claim set forth below are, therefore, defined in this specification to include not only the combination of elements, steps or limitations which are literally set forth, but all equivalent structure, material or acts for performing substantially the same function in substantially the same way to obtain substantially the same result. In this sense it is therefore contemplated that an equivalent substitution of two or more elements, steps and/or limitations may be made for any one of the elements, steps or limitations in a claim set forth below or that a single element, step or limitation may be substituted for two or more elements, steps and/or limitations in such a claim. Although elements, steps or limitations may be described above as acting in certain combinations and even initially claimed as such, it is to be expressly understood that one or more elements, steps and/or limitations from a claimed combination can in some cases be excised from the combination and that the claimed combination may be directed to a sub-combination or variation of a sub-combination. As such, notwithstanding the fact that the elements, steps and/or limitations of a claim are set forth below in a certain combination, it must be expressly understood that the invention includes other combinations of fewer, more or different elements, steps and/or limitations, which are disclosed in above combination even when not initially claimed in such combinations. Furthermore, insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements. Accordingly, the claims are thus to be understood to include what is specifically illustrated and described above, what is conceptually equivalent, what can be obviously substituted and also what essentially incorporates the essential idea of the invention.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as, e.g., “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising”, variations thereof such as, e.g., “comprise” and “comprises”, and equivalent open-ended transitional phrases thereof like “including”, “containing” and “having”, encompass all the expressly recited elements, limitations, steps, integers, and/or features alone or in combination with unrecited subject matter; the named elements, limitations, steps, integers, and/or features are essential, but other unnamed elements, limitations, steps, integers, and/or features may be added and still form a construct within the scope of the claim. Specific embodiments disclosed herein may be further limited in the claims using the closed-ended transitional phrases “consisting of” or “consisting essentially of” (or variations thereof such as, e.g., “consist of”, “consists of”, “consist essentially of”, and “consists essentially of”) in lieu of or as an amendment for “comprising.” When used in the claims, whether as filed or added per amendment, the closed-ended transitional phrase “consisting of” excludes any element, limitation, step, integer, or feature not expressly recited in the claims. The closed-ended transitional phrase “consisting essentially of” limits the scope of a claim to the expressly recited elements, limitations, steps, integers, and/or features and any other elements, limitations, steps, integers, and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps, integers, and/or features specifically recited in the claim, whereas the meaning of the closed-ended transitional phrase “consisting essentially of” is being defined as only including those elements, limitations, steps, integers, and/or features specifically recited in the claim and those elements, limitations, steps, integers, and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such, the embodiments described herein or so claimed with the phrase “comprising” expressly and unambiguously provide description, enablement, and support for the phrases “consisting essentially of” and “consisting of.”

Lastly, all patents, patent publications, and other references cited and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard is or should be construed as an admission that the inventors are not entitled to antedate such disclosure by virtue of prior invention or for any other reason. All statements as to the date or representation as to the contents of these documents are based on the information available to the applicant and do not constitute any admission as to the correctness of the dates or contents of these documents.

1. A composition comprising one or more monoterpene compounds and HA polymers.

2. The composition of claim 1, wherein the one or more monoterpene compounds include a monoterpene, a derivative of a monoterpene, a monoterpenoid, a derivative of a monoterpenoid, or any combination thereof.

3. The composition of claim 2, wherein the monoterpene is a linear monoterpene, a monocyclic monoterpene, a bicyclic monoterpene, a tricyclic monoterpene, or any combination thereof.

4. The composition of claim 2, wherein the derivative of a monoterpene is a carbamate, an ester, an ether, an alcohol or an aldehyde of a linear monoterpene, a carbamate, an ester, an ether, an alcohol or an aldehyde of a monocyclic monoterpene, a carbamate, an ester, an ether, an alcohol or an aldehyde of a bicyclic monoterpene, a carbamate, an ester, an ether, an alcohol or an aldehyde of a tricyclic monoterpene, or any combination thereof.

5. The composition of claim 3, wherein the linear monoterpene is α-myrcene, β-myrcene, α-ocimene, β-ocimene, or any combination thereof.

6. The composition of claim 3, wherein the monocyclic monoterpene is limonene, α-phellandrene, β-phellandrene, α-terpineol, β-terpineol, γ-terpineol, 4-terpineol, terpinen-4-ol, α-terpinene, β-terpinene, γ-terpinene, δ-terpinene (terpinolene), or any combination thereof.

7. The composition of claim 3, wherein the bicyclic monoterpene is camphene, carene, myrtersol, myrtenal, α-pinene, β-pinene, pinocarveol, sabinene, thujene, verbanol, verbanon, or any combination thereof.

8. The composition of claim 3, wherein the tricyclic monoterpene include tricyclene.

9. The composition of claim 2, wherein the monoterpenoid is a linear monoterpenoid, a monocyclic monoterpenoid, a bicyclic monoterpenoid, a tricyclic monoterpenoid, or any combination thereof.

10. The composition of claim 2, wherein the derivative of a monoterpenoid is a carbamate, an ester, an ether, an alcohol or an aldehyde of a linear monoterpenoid, a carbamate, an ester, an ether, an alcohol or an aldehyde of a monocyclic monoterpenoid, a carbamate, an ester, an ether, an alcohol or an aldehyde of a bicyclic monoterpenoid, a carbamate, an ester, an ether, an alcohol or an aldehyde of a tricyclic monoterpenoid, or any combination thereof.

11. The composition of claim 9, wherein the linear monoterpenoid is citral, citronellal, citronellol, geranial (citral A), geraniol, geranyl pyrophosphate, halomon, linalool, myrcenol, nerol (citral B), or any combination thereof.

12. The composition of claim 9, wherein the monocyclic monoterpenoid is carvacrol, p-cymene, grapefruit mercaptan, menthol, perillyl alcohol, α-thujaplicin, β-thujaplicin (hinokitiol), γ-thujaplicin, thymol, or any combination thereof.

13. The composition of claim 9, wherein the bicyclic monoterpenoid is ascaridole, borneol, bornyl acetate, camphor, eucalyptol, umbellulone, or any combination thereof.

14. The composition of claim 1, wherein the HA polymers comprise low molecular weight HA polymers, high molecular weight HA polymers, or both low molecular weight HA polymers and high molecular weight HA polymers.

15. The composition of claim 14, wherein when both low molecular weight HA polymers and high molecular weight HA polymers, the weight ratio of high molecular weight HA polymers to low molecular weight HA polymers is about 20, about 15, about 10, about 5, about 1, about 0.07, about 0.05, about 0.2, or about 0.1 to 1.

16. The composition of claim 1, wherein the HA polymers comprise uncrosslinked HA polymers, crosslinked HA polymers, or both uncrosslinked HA polymers and crosslinked HA polymers.

17. The composition of claim 16, wherein the uncrosslinked HA polymers comprise low molecular weight uncrosslinked HA polymers, high molecular weight uncrosslinked HA polymers, or both low molecular weight uncrosslinked HA polymers and high molecular weight uncrosslinked HA polymers.

18. The composition of any one of claim 16, wherein the crosslinked HA polymers comprise low molecular weight crosslinked HA polymers, high molecular weight crosslinked HA polymers, or both low molecular weight crosslinked HA polymers and high molecular weight crosslinked HA polymers.

19. The composition of claim 16, wherein when both low molecular weight crosslinked HA polymers and high molecular weight HA polymers are present, low molecular weight crosslinked HA polymers are crosslinked to low molecular weight crosslinked HA polymers, high molecular weight crosslinked HA polymers are crosslinked to high molecular weight crosslinked HA polymers, low molecular weight crosslinked HA polymers are crosslinked to high molecular weight crosslinked HA polymers, or any combination thereof.

20. The composition of claim 1, wherein the composition further comprises one or more monoterpene compound-HA polymer conjugates, wherein each of the one or more monoterpene compound-HA polymer conjugates comprises a monoterpene compound covalently linked to a HA polymer.
